(19) United States
(12) Patent Application Publication
(10) Pub. No.: US 2021/0356472 A1
(43) Pub. Date: Nov. 18, 2021

(54) METHODS AND COMPOSITIONS FOR DETECTION AND VISUALIZATION OF OXIDATIVE STRESS-INDUCED CARBONYLATION IN CELLS

(71) Applicant: The Research Foundation for The State University of New York, Binghamton, NY (US)

(72) Inventors: Susan Bane Tuttle, Vestal, NY (US); Kamalika Mukherjee, Binghamton, NY (US)

(21) Appl. No.: 17/321,088

(22) Filed: May 14, 2021

Related U.S. Application Data
(60) Provisional application No. 63/025,852, filed on May 15, 2020.

(51) Int. Cl. G01N 33/58; G01N 33/574; G01N 33/50; G01N 21/64

(52) U.S. Cl. CPC G01N 33/582; G01N 33/57496; G01N 33/5014; G01N 21/6458; G01N 21/6402; G01N 2201/06113; G01N 2800/7009; G01N 2800/7042; G01N 2800/709; G01N 2021/6439; G01N 21/6428

(57) ABSTRACT

Oxidative stress (OS) is associated with a wide variety of diseases and disorders. Detection of oxidative stress in living systems typically relies on fluorescent probes for reactive oxygen species (ROS), which is challenging because of their short life span and high reactivity. OS-induced biomolecule carbonylation is a stable modification that also possesses a chemically reactive functional group, and may be detected with a hydrazine, alkoxyamine or hydrazide-containing probe, in a hydrazone or oxime-forming reaction, that does not require strong acid catalysis or nucleophilic catalysis with an aromatic amine. Fluorophores possessing hydrazine, alkoxyamine or hydrazide functional groups can undergo reaction with carbonylated biomolecules in live cells, fixed cells, and tissue sample, and these products can be observed using fluorescence microscopy.

### CROSS REFERENCE TO RELATED APPLICATIONS

The present application is a non-provisional of, and claims benefit of priority under 35 U.S.C. § 119(e) from U.S. Provisional Patent Application No. 63/025,852, filed May 15, 2021, the entirety of which is expressly incorporated herein by reference.

### STATEMENT OF GOVERNMENT SUPPORT

This invention was made with government support under R15-CA227747 and R15-GM102867 awarded by the National Institutes of Health. The government has certain rights in the invention.

### FIELD OF THE INVENTION

The present invention relates to the field of reagents for detection of oxidative stress in cells, and more particularly to a hydrazine fluorophore for detection of biomolecule carbonylation.

### BACKGROUND OF THE INVENTION

Each patent, patent application, and non-patent reference cited herein is expressly incorporated herein in its entirety, for all purposes.

A myriad of biomarkers containing aldehyde and ketone moieties exist and can play an important role in the biological, forensic, medical and industrial sciences. In particular, aldehydes and ketones are known to be key end products in the degradation of a variety of biological molecules, such as, lipids, nucleic acids, carbohydrates and proteins. In a number of instances, these end products are a result of oxidative stress. In one example, malondialdehyde and 4-hydroxynonenal are markers for lipid peroxidation.

A number of reagents for the detection of aldehyde and ketone-containing moieties have been proposed, however, each with limited success. Among the most widely used of such reagents are dansyl hydrazine, fluorescein thiosemicarbazide, various biotin hydrazides, biotin hydroxylamine (ARP), and various aromatic amines (2-aminopyridine, 8-aminonaphthalene-1,3,6-disulfonic acid, 1-aminopyrene-3,6,8-trisulfonic acid, 2-aminoacridone). Unfortunately, these reagents require additional purification and/or secondary reagents.

Existing methods of labeling carbohydrates that utilize hydrazine, hydroxylamine and amine derivatization reagents have focused on labeling aldehydes present in, or introduced into, carbohydrates, particularly the so-called “reducing sugars”. Aldehydes are typically introduced into carbohydrates by periodate oxidation. The adduct formed with the reducing sugar can be further stabilized by treatment with borohydride or a cyanoborohydride. The derivatization reaction typically proceeds or is followed by a separation technique such as chromatography, electrophoresis, precipitation, affinity isolation or other means before direct or indirect detection of the labeled product. Unlike the foregoing, which require purification and the use of a secondary detection reagent, the reagents of the present invention permit rapid in-situ detection of aldehyde and ketone moieties upon contact.

U.S. Pat. No. 6,967,251 (Haugland et al.) describes aniline-substituted quinazolinone compounds which can display fluorescent changes upon binding of aldehyde-containing compounds in a gel.

A family of hydrazinyl substituted xanthene dyes have been previously described in U.S. Pat. No. 4,420,627 (Widiger). However, each of the xanthene moieties in Widiger are substituted to prevent analyte binding of the molecule. Particularly, the terminal nitrogen atom in the hydrazinyl moiety in Widiger is carbonylated, such that the hydrazinyl group is not nucleophilic and unable to bind an aldehyde or ketone moiety. Accordingly, the compounds are not functional in carbonyl reaction.

Oxidative stress (OS) is associated with a wide variety of diseases and disorders. Detection of oxidative stress in living systems typically relies on fluorescent probes for reactive oxygen species (ROS), which is challenging because of their short life span and high reactivity. Oxidative damage caused by OS produces a more stable signal, but these biomarkers are usually detected using techniques that are not compatible with live cells. OS-induced biomolecule carbonylation is a stable modification that also possesses a chemically reactive functional group, and its detection typically employs a chemical reaction with a hydrazine-containing probe within the process. These hydrazone-forming reactions require strong acid catalysis or nucleophilic catalysis with an aromatic amine when performed on isolated biomaterial or on fixed cells.

Redox homeostasis is essential for normal cellular functions. An imbalance marked by an enhanced level of reactive oxygen species (ROS) is referred to as oxidative stress (OS). OS is emblematic of a wide variety of disorders and is implicated in diseases such as diabetes, cancer and neurodegenerative disorders and in conditions varying from normal aging to xenobiotic toxicity (Dalle-Donne, Giustarini, Colombo, Rossi, & Milzani, 2003; Dalle-Donne, Rossi,

Colombo, Giustarini, & Milzani, 2006; Ramana, Srivastava, & Singhal, 2014; Thanan et al., 2014).

Detection of OS in situ is often accomplished by trapping the ROS with an exogenous molecule that will undergo a change in properties upon chemical reaction with the short-lived species (Zhang, Dai, & Yuan, 2018). Fluorescent proteins engineered to detect ROS have been developed that can be used for imaging in vivo in addition to intracellular imaging (Wang et al., 2013). Under certain circumstances, temporal observation of ROS production is possible with these methods.

Alternatively, oxidative damage that is a result of OS may be employed as a more stable signal for visualization (Katerji, Filippova, & Duerksen-Hughes, 2019). Carbonylation is an irreversible consequence of OS and carbonylated biomolecules are widely used biomarkers. Carbonylation is an attractive observable because OS-induced carbonyls are widely distributed across cellular compartments and biomolecules. For example, lipid peroxidation is prevalent during OS, yielding products such as 4-hydroxynonenal, malondialdehyde, and acrolein, which can be membrane-associated or appended to other biomolecules such as proteins and DNA (Halliwell & Chirico, 1993). Direct oxidation of DNA can produce the mutagenic adduct 8-hydroxy-2′-deoxyguanosine (8-OHDG), which enolizes to 8-oxo-2′-deoxyguanosine, and abasic sites in nucleic acid polymers (Marnett, 2000). Protein carbonyls can be generated either by direct oxidation of amino acid side chains (lysine, arginine, proline and threonine) or by addition of oxidized sugars and lipids to the protein (Dalle-Donne, Aldini, et al., 2006).

Another attractive feature of carbonylation as a biomarker is that the covalent modification possesses a chemically reactive group that is orthogonal to endogenous functional groups. It has long been known that hydrazines and alkoxyamines undergo condensation reactions with aldehydes and ketones to form reversible but stable products (hydrazones and oximes, respectively) (Kölmel & Kool, 2017). Levine et al. introduced 2, 4-dinitrophenylhydrazine (DNPH) as the first viable biomolecule-carbonyl detection tool 30 years ago (Levine et al., 1990); the general procedure remains widely used for carbonyl detection (Colombo et al., 2016; Wehr & Levine, 2013). Variations of this overall approach have been applied to samples ranging from patient serum and urine to research models (cells, tissues, whole organism) (Chaudhuri, Wei, Bhattacharya, & Hamilton, 2015; Frohnert & Bernlohr, 2013; Kuzmic et al., 2016; Tamarit et al., 2012; Ugur, Coffey, & Gronert, 2012; Weber, Davies, & Grune, 2015; Wehr & Levine, 2013; L. J. Yan & M. J. Forster, 2011).

The most common methods used to evaluate carbonylation in cell lysate and its components are still variations of the DNPH-based assay (L.-J. Yan & M. J. Forster, 2011). It is typically performed in strong acid, which prevents the method from being applied to delicate or living systems. An advantage of the DNPH assay is that it is simple, requires inexpensive detection systems (an absorption spectrophotometer) and is reasonably sensitive. The availability of anti-dinitrophenyl antibodies allows for detection of carbonylation using ELISA and Western blot and isolation using immunoaffinity methods. DNPH has also been used prior to mass spectrometry analysis of carbonylated proteins.

Other reagents that are used for carbonyl detection employ the same type of chemical reaction as DNPH. Biotin hydrazide is particularly useful for separations of carbonylated biomolecules from complex mixtures, which are often subjected to mass spectrometry-based techniques to enable identification of specific proteins and sites of carbonylation on those proteins (Madian & Regnier, 2010). Fluorescent hydrazines, hydrazides and related reactive groups provide a sensitive direct read of carbonylated biomolecules.

Intracellular localization of carbonylated biomolecules is possible by performing immunocytochemistry following the DNPH reaction or by directly conjugating carbonyl-reactive fluorophores. This allows for visualization (microscopy) or quantification (flow cytometry) of the global distribution of cellular carbonyls in situ. It is also possible to localize multiple biomarkers in the same sample by using spectrally distinct fluorescent labels. Multiplexing makes it feasible to simultaneously investigate cross-talk between physiologically relevant cellular events.

Intact cell analysis can be performed either in fixed (aldehyde or methanol) or live cells. Fixed cell analysis, whether by immunocytochemistry or by direct fluorophore labeling, enables observation of the spatial distribution and evaluation of the global status of cellular carbonyls. Live cell analysis allows both spatial and temporal resolution, which may be essential to decipher molecular mechanisms underlying clinical manifestations of biomolecule carbonylation.

A number of reagents for the detection of aldehyde and ketone-containing moieties have been proposed, however, each with limited success. Among the most widely used of such reagents are dansyl hydrazine, fluorescein thiosemicarbazide, various biotin hydrazides, biotin hydroxylamine (ARP), and various aromatic amines (2-aminopyridine, 8-aminonaphthalene-1,3,6-disulfonic acid, 1-aminopyrene-3,6,8-trisulfonic acid, 2-aminoacridone). Unfortunately, these reagents require additional purification and/or secondary reagents. See, WO2009094536; U.S. Pat. Nos. 6,967,251; and 4,420,627.

Wang et al (2019) teaches a two-photon fluorescence probe 5-methyl-2-(2-oxo-4-(trifluoromethyl)-2H-chromen-7-yl)-2,4-dihydro-3H-pyrazol-3-one (MD-B), for imaging .OH in living systems. MD-B does not, however, image biological macromolecules.

U.S. 2004/0171635 provides hydrazone-based fluorescent and pro-fluorescent reagents and linkers, including conjugationally extended hydrazine compositions, fluorescent hydrazone compositions, methods of the formation of hydrazones from the reaction of conjugationally extended hydrazines with conjugationally extended carbonyls. The use of various fluorescent hydrazides, thiosemicarbazides and hydrazides are used to detect aldehydes on biological molecules. For example, Ahn et al. (B. Ahn, S. G. Rhee and E. R. Stadtman, Anal. Biochem. 161:245 (1987) describe the use of fluorescein hydrazide and fluorescein thiosemicarbazide for the fluorometric determination of protein carbonyl groups and for the detection of oxidized proteins on polyacrylamide gels. Proudnikov and Mirzabekov (Nucl. Acids Res. 24:4535 (1996)) describe labeling of DNA and RNA to identify acid-induced depurination that results in production of aldehyde moieties detected by reaction of fluorescent labels containing hydrazide groups in the presence of sodium cyanoborohydride. Others have labeled the reducing end of polysaccharides with fluorescent hydrazides. These methods are used to detect aliphatic aldehyde groups on biomolecules. The fluorescent moiety is incorporated on the hydrazine or hydrazide that forms a hydrazone on reaction with the aldehyde present on the biomolecule. Hydrazones formed between certain aromatic aldehydes and aromatic hydrazines and not aromatic hydrazides or aromatic thiosemicarbazides form fluorescent molecules (J. Wong and F. Bruscato, Tet. Lett. 4593, 1968). Hydrazones formed specifically from 2-substituted aldehyde heterocycles and 2-substituted hydrazine heterocycles become fluorescent on chelation to zinc (D. E. Ryan, F. Snape and M. Winpe, Anal. Chim. Acta 58:101, 1972).

### SUMMARY OF THE INVENTION

In live cells, hydrazone-forming reactions are surprisingly facile. Fluorophores possessing hydrazine or hydrazide functional groups can undergo reaction with carbonylated biomolecules in live cells, and these products can be observed using fluorescence microscopy.

OS-induced biomolecule carbonyls can be visualized in live cells following direct conjugation with a hydrazine-functionalized fluorophore, 7-hydrazinyl-4-methylcoumarin (coumarin hydrazine, CH), to cellular carbonyls (Mukherjee, Chio, Sackett, & Bane, 2015). Benzocoumarin hydrazine (BzCH), a hydrazine-functionalized fluorophore (Mukherjee et al., 2017), allows visualization of carbonyls in live cells using a one-step assay. An early response to chemical toxicity is oxidative stress (OS), which is evidenced by an upsurge of oxidants, such as reactive oxygen species (ROS), and depletion of reductants, such as reduced glutathione (GSH). While changes in ROS and GSH are transient, a cardinal irreversible consequence of OS is the carbonylation of biomolecules (FIG. 21A). Carbonylated biomolecules may thus serve as an early stable biomarker for screening potential nephrotoxins. In general, biomolecule-carbonyls are detected colorimetrically or fluorescently by hydrazine- or hydrazide-functionalized reporter molecules (Katerji, M., Filippova, M. & Duerksen-Hughes, P. Approaches and Methods to Measure Oxidative Stress in Clinical Samples: Research Applications in the Cancer Field. Oxid Med Cell Longev 2019, 1279250 (2019); Kuzmic, M. et al. In situ visualization of carbonylation and its co-localization with proteins, lipids, DNA and RNA in Caenorhabditis elegans. Free Radic Biol Med 101, 465-474 (2016).). These routinely used assays involving immunocytochemistry or immunochemical/chemical analysis are lengthy, tedious and mostly enable end-point measurements. The present technology extrapolates from probes developed for assessing biomolecule-carbonyls in live cells (Mukherjee, K. et al. Benzocoumarin Hydrazine: A Large Stokes Shift Fluorogenic Sensor for Detecting Carbonyls in Isolated Biomolecules and in Live Cells. ACS Sens 2, 128-134 (2017); Mukherjee, K., Chio, T. I., Sackett, D. L. & Bane, S. L. Detection of oxidative stress-induced carbonylation in live mammalian cells. Free Radic Biol Med 84, 11-21 (2015).). The present technology provides a fluorescent sensor and a simple, highly sensitive assay for screening nephrotoxins that exploit biomolecule carbonylation as an early sign of cellular damage.

TFCH is a fluorescent probe for oxidative-stress induced carbonylation in live cells. FIG. 21A shows a schematic representation of fluorogenic detection of oxidative stress-induced carbonylation. FIG. 21B shows the chemical structure of 4-trifluoromethyl-7-hydrazinyl-2H-chromen-2-one (TFCH) and its corresponding hydrazone (TFCZ). FIG. 21C shows emission spectra of 10 82 M TFCH or TFCZ in phosphate buffer containing 0.5% (v/v) DMSO. Excitation wavelength=405 nm. FIG. 21D shows A549 cells grown in standard media (control) or serum-free media (SFM) for 24 h were allowed to react with 20 82 M TFCH for 30 min, washed, and imaged live. FIG. 21E shows bar graphs showing quantification of cellular carbonyls detected by TFCH in live control and serum-starved A549 cells. In the experiments reported in FIG. 21E, TFCH (20 82 M) was added to live cells and incubated for 30-60 min, which were then rinsed, fixed and imaged. At least three independent experimental sets were performed, and fluorescence associated with >100 cells were quantified. Error bars represent SEM.

In the process of designing the probe's scaffold, the substitution of coumarin with a 4-trifluoromethyl group was found to lower the bleaching coefficient, providing a desirable attribute for optical applications. (Schill, H. et al. 4-Trifluoromethyl-substituted coumarins with large Stokes shifts: synthesis, bioconjugates, and their use in super-resolution fluorescence microscopy. Chemistry 19, 16556-16565 (2013).). 4-trifluoromethyl-7-hydrazinyl-2H-chromen-2-one (TFCH) was therefore synthesized, which possesses a hydrazine functionality to endow reactivity to carbonyls. Indeed, TFCH covalently conjugates with carbonyl functional groups to form hydrazones (TFCZ's) (FIG. 21B). In particular, hydrazone formation with an aliphatic aldehyde (i.e., propanal) resulted in a ˜4-fold increase in probe fluorescence in buffered aqueous solution (phosphate buffer, pH 7.0 with 0.5% DMSO), indicating its suitability for detection of carbonyls under physiological conditions (FIG. 21C). The limit of detection (LOD) of TFCZ is ˜89 nM in cell lysate (of A549 lung cancer cells), which demonstrates its high sensitivity in the biological milieu (FIGS. 23A and 23B. The LOD of TFCZ is an order of magnitude smaller than that of our previously reported fluorophore. (Mukherjee, K. et al. Benzocoumarin Hydrazine: A Large Stokes Shift Fluorogenic Sensor for Detecting Carbonyls in Isolated Biomolecules and in Live Cells. ACS Sens 2, 128-134 (2017)). Together, these data strongly support the idea that TFCH can serve as a highly sensitive and photochemically desirable reporter molecule for the detection of various carbonylated biomolecules.

Oxidized BSA was used as a model carbonylated biomolecule and TFCH's ability to detect it in an isolated system was tested. TFCH was able to fluorescently label oxidized BSA and form a stable conjugate (FIG. 21C). Next, the compatibility of TFCH for cell-based assays was established using lung cancer (A549) cells as a model cell line. TFCH's influx and efflux were achieved readily (<5 min) (FIGS. 21D and 21E). Using serum-free media (SFM) as an OS-induction model, TFCH was shown to be usable to perform a facile platereader-based assay suitable for high throughput screening (HTS) (FIGS. 22A, 22B). Importantly, given the turn-up property of the fluorophore, it was possible to identify carbonyls in live cells in the presence of unreacted fluorophores by a one-step high content screening (HCS)-compatible assay. Low concentration of the dye (2 82 M) can detect carbonyls induced by serum starvation in live cells within 30 min (FIG. 22C). This single-step (no wash) assay format may be particularly suitable for screening molecules that have a propensity to induce cell detachment. Alternatively, excess fluorophore can be removed and the cells can be either imaged live (FIGS. 21D and 21E) or can be fixed and preserved for analysis at a later time Indeed, a similar spatial distribution and increase in carbonylation due to serum starvation was detected in both live (FIGS. 5, 19B) and fixed cells (fixed after fluorescent labeling in live cells). Together these data establish TFCH as a tool for visualizing and quantifying biomolecule carbonyls in live cells. Three commercially available hydrazide fluorophores, 7-(diethylamino)coumarin-3-carbohydrazide (DCCH), Texas Red® hydrazide (TxRH) and BODIPY™ hydrazide (BODIPYH), were assessed, and found suitable as optical probes for carbonylation in live cells. See also, (Vemula, Ni, & Fedorova, 2015).

In general, intracellular carbonylation is detected in cell lysate or in intact cells. An account of common reagents and methods for carbonylation detection is shown in FIG. 1. In cell lysate, standard protocols tend to focus on carbonylated proteins (Colombo et al., 2016; L. J. Yan & M. J. Forster, 2011). Oxidized lipids, which are large contributors to the total cellular carbonylation, are detected in whole cell lysate as free lipid and as protein conjugates (Hauck & Bernlohr, 2016), but quantitative analysis is typically performed on protein fractions. The hydrazines and hydrazides undergo unexpectedly fast reaction with carbonyls in live cells. Hydrazide-containing reagents are notoriously slow to react with aldehydes and especially ketones at physiological pH (Kolmel & Kool, 2017); however, in cells these reactions rates are increased by orders of magnitude. See,

DNPH: (Buss, Chan, Sluis, Domigan, & Winterbourn, 1997; Mesquita et al., 2014; Wehr & Levine, 2013; L. J. Yan & M. J. Forster, 2011)

BH: (Coffey & Gronert, 2016; Yoo & Regnier, 2004)

ARP: (Chung, Miranda, & Maier, 2008)

Fluorophore: Rhodamine Hz (20) (Georgiou et al., 2018; Mukherjee et al., 2015; Tamarit et al., 2012)

Fixed cells: FACS with DNPH-(Talarico et al., 2016),

Direct fluorophore: Coumarin Hz (Vemula et al., 2015),

Immunocytochemistry: OxyICC (Nascimento et al., 2015), DNP AB (Lucero et al., 2008)

Intact Cells: Coumarin Hz (Vemula et al., 2015), CH, BzCH (Mukherjee et al., 2017; Mukherjee et al., 2015)

Various fluorophores are known. In general, the fluorophores according to the present invention form hydrazones and oximes with aldehydes and ketones, that are stable after formation, form at physiological conditions (e.g., pH (6-8.5), temperature (20 C-45 C)), are compatible with use on living cells or tissue (at least at the time of formation of the hydrazone or oxime), have a sufficient fluorescent peak shift to differentiate between bound and free fluorophore. In some cases, the molecule, prior to reaction, is not fluorescent, or has a fluorescence which is not detectable using the equipment used to detect or image the bound dye. The fluorophores may be pH insensitive, though in some cases pH sensitivity may be exploited to determine not only location of carbonyls, but also compartment conditions. The fluorophores are especially suitable for staining insoluble or macromolecular cellular components that have formation of ketones and aldehydes as a result of hydroxyl radical interaction, typically resulting from oxidative stress. The fluorescent structure of the dye may be, for example, a coumarin, fluorescein, rhodamine, or Texas Red ® (sulforhodamine 101) type structure, having an available hydrazine or hydrazide ligand.

The fluorophore may have a trifluoro-alkyl (e.g., trifluoromethyl) substituent which is both electron withdrawing and typically renders the molecule lipophilic. See Huchet, Quentin A., Bernd Kuhn, Björn Wagner, Holger Fischer, Manfred Kansy, Daniel Zimmerli, Erick M. Carreira, and Klaus Muller. “On the polarity of partially fluorinated methyl groups.” Journal of Fluorine Chemistry 152 (2013): 119-128. Based on a simple C—F bond vector analysis, the polarity increase upon exchange of a terminal methyl by a trifluoromethyl group in an aliphatic substituent should be comparable to that arising from a single H/F exchange at the terminal methyl group, while the volume increase in the former case is three times larger than that in the latter. This may explain the observation that replacement of a methyl by a fluoromethyl group results in a more pronounced lowering of molecular lipophilicity than its replacement by a trifluoromethyl group. See also, Muller, Norbert. “When is a trifluoromethyl group more lipophilic than a methyl group? Partition coefficients and selected chemical shifts of aliphatic alcohols and trifluoroalcohols.” Journal of pharmaceutical sciences 75, no. 10 (1986): 987-991; Koksch, Beate, Norbert Sewald, Hans-Dieter Jakubke, and Klaus Burger. “Synthesis and incorporation of a-trifluoromethyl-substituted amino acids into peptides.” 1996.; Sewald, Norbert, W. Hollweck, K. Mütze, C. Schierlinger, L. C. Seymour, K. Gaa, K. Burger, B. Koksch, and H. D. Jakubke. “Peptide modification by introduction of α-trifluoromethyl substituted amino acids.” Amino Acids 8, no. 2 (1995): 187-194; Tempesti, Tomas C., M. Gabriela Alvarez,

Marcelo Francisco de Araújo, Francisco Eduardo Aragao Catunda Junior, Mário Geraldo de Carvalho, and Edgardo N. Durantini. “Antifungal activity of a novel quercetin derivative bearing a trifluoromethyl group on Candida albicans.” Medicinal Chemistry Research 21, no. 9 (2012): 2217-2222. Thus, it would be expected that TFCH would be less water-soluble than CH, and therefore that it would yield inferior sensitivity under confocal microscopy to oxidative stress-induced carbonylation of proteins, carbohydrates and nucleic acids. Jensen, Ellen C. “Use of fluorescent probes: their effect on cell biology and limitations.” The Anatomical Record: Advances in Integrative Anatomy and Evolutionary Biology 295, no. 12 (2012): 2031-2036. Bouvrais H, Pott T, Bagatolli L A, Ipsen J H, Méléard P. 2010. Impact of membrane-anchored fluorescent probes on the mechanical properties of lipid bilayers. Biochim Biophys Acta 1798: 1333-1337; However, the opposite was observed in practice.

The fluorophore may be a Resazurin/resorufin derivative, though this may render the fluorophore concurrently sensitive to cell viability. This may be of interest, for example, where one seeks to understand sublethal ROS in cells.

When measured in cell-free systems, the reaction of TFCH with biomolecule ketones was eight hundred times slower than in living cells, and was sufficiently slow to predict a poor outcome if the TFCH was to be used to label biomolecules that have carbonyls resulting from reactive oxygen species (ROS). However, when tested, it was found that in living cells, the reaction speed of TFCH with carbonylated biomolecules was fast, and further that the limit of detection (LOD) was lower than for BzCH, providing a more sensitive test. It is believed that, in vivo, small molecules such as amino acids are involved in catalysis of the reaction.

Likewise, the fluorophore may have other chemical or biological characteristics which promote binding to certain structures within a cell, a tissue, or which increase signal or detection sensitivity by reducing interference or non-specific or off-target binding.

In some cases, the fluorophore may provide fluorescence (Forster) resonant energy transfer (FRET) with another fluorescent due or structure. For example, if the carbonyl-containing molecule is a nucleic acid, a fluorescent probe may be used to detect a nearby nucleic acid sequence, with FRET used to distinguish locations where both the hydrazine or hydrazide dye, and the fluorescent probe, are both localized. Similarly, a fluorescently-labelled antibody (or other structurally-specific binding peptide or other molecule) may also be involved in FRET with the hydrazine or hydrazide dye.

In other cases, duplex or triplex labelling of cells or tissues may be performed in separate fluorescence channels of a detector or microscope. For example, other dyes that assess other aspects of oxidative metabolism may be used. Note that other types of differentiation between free and bound, or cellular compartment, may be employed other than fluorescent wavelength. See, 20020055133; 20120252699.

The dye may be a proposed dye with a hydrazine or hydrazide functionality, or a modified dye core. See, U.S. Pat. Nos. 10,215,751; 10,139,400; 10,071,990; 10,053,484; 10,018,617; 9,983,211; 9,951,271; 9,951,227; 9,850,383; 9,791,450; 9,701,841; 9,579,402; 9,346,957; 9,127,164; 9,097,667; 9,056,885; 9,006,437; 8,981,100; 8,816,095; 8,791,258; 8,785,119; 8,735,601; 8,658,434; 8,436,170; 8,329,413; 8,318,953; 8,303,936; 8,252,530; 8,148,423; 8,114,904; 8,030,096; 7,910,319; 7,871,773; 7,795,042; 7,635,598; 7,597,878; 7,507,395; 7,504,496; 7,226,740; 6,800,765; 6,740,497; 6,664,047; 6,225,050; 6,191,278; 6,048,982; 5,877,310; 5,658,751; 20190185434; 20180328931; 20180238912; 20170108518; 20160356782; 20160139158; 20150141282; 20150079622; 20150037834; 20140356894; 20140220560; 20130317207; 20130288911; 20130102021; 20110053162; 20110136201; 20100291547; 20100278745; 20100015054; and 20080274907.

Dyes of the invention may be particularly useful for use with commercially equipped excitation sources and detectors. For example, the 405 nm diode laser line excitation source is common in most major fluorescence-based life science instruments due to its reliability and low cost.

Definitions

The below definitions apply both directly to the explicit disclosure hereof, and also to supplement disclosure of the incorporated references, as consistent or combinable with the express disclosure herein.

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119 1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable (e.g. R, L, (R₁)ₐ, (L)_{q}) occurs more than one time in any constituent, its definition on each occurrence is independent at every other occurrence. Combinations of substituents and variables are permissible only if such combinations result in stable compounds.

Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only. Substitution of a ring by a substituent generally allows the substituent to be a cyclic structure fused to the ring.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched, straight-chain, and cyclic saturated aliphatic hydrocarbon groups. Alkyl groups specifically include methyl, ethyl, propyl, butyl, pentyl, hexyl, heptyl, octyl, nonyl, decyl, and so on, as well as cycloalkyls such as cyclopropyl, cyclobutyl, cyclopentyl, cyclohexyl, tetrahydronaphthalene, methylenecylohexyl, and so on. “Alkoxy” represents an alkyl group attached through an oxygen bridge.

The term “alkenyl” refers to a non-aromatic hydrocarbon group, straight, branched or cyclic, containing at least one carbon to carbon double bond. Alkenyl groups include, but are not limited to, ethenyl, propenyl, butenyl and cyclohexenyl. The straight, branched or cyclic portion of the alkenyl group may contain double bonds and may be substituted if a substituted alkenyl group is indicated.

The term “alkynyl” refers to a hydrocarbon group, straight, branched or cyclic, containing at least one carbon to carbon triple bond. Alkynyl groups include, but are not limited to, ethynyl, propynyl and butynyl. The straight, branched or cyclic portion of the alkynyl group may contain triple bonds and may be substituted if a substituted alkynyl group is indicated.

As used herein, “aryl” is intended to mean any stable monocyclic or polycyclic carbon ring of up to 7 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic or bicyclic ring of up to 7 atoms in each ring, wherein at least one ring is aromatic and contains from 1 to 4 heteroatoms selected from the group consisting of 0, N and S. Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, pyrazinyl, pyridazinyl, pyridinyl, pyrimidinyl, pyrrolyl, tetrahydroquinoline, xanthenyl, and coumarinyl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing at least one heteroatom which is O, N or S. This definition includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

The alkyl, alkenyl, alkynyl, cycloalkyl, aryl, heteroaryl and heterocyclyl substituents may be unsubstituted or unsubstituted, unless specifically defined otherwise. For example, an alkyl group may be substituted with one or more substituents selected from OH, oxo, halo, alkoxy, dialkylamino, or heterocyclyl, such as morpholinyl or piperidinyl.

The terms “halo” or “halogen” are intended to include chloro, fluoro, bromo and iodo groups.

The term “aromatic” is used in its usual sense, including unsaturation that is essentially delocalized across multiple bonds, such as around a ring.

The term “substituent” refers to an atom, radical or chemical group which replaces a hydrogen in a substituted chemical group, radical, molecule, moiety or compound.

“Spiro” as used herein, refers to a cylic moiety which is attached to another group such that one of the ring atoms of the cyclic moiety is also an atom of said other group. A non-spiro substituent is a moiety cyclic or noncylic which is directly attached to said other group via bond connection between atoms of the non-spiro moiety and said other group.

Unless otherwise stated, the term “radical”, as applied to any molecule or compound, is used to refer to a part, fragment or group of the molecule or compound rather than to a “free radical”. A radical may be linked to another moiety through a covalent bond.

When a group or moiety can be substituted, the term “substituted” indicates that one or more hydrogens on the group indicated in the expression using “substituted” can be replaced with a selection of recited indicated groups or with a suitable group known to those of skill in the art (e.g., one or more of the groups recited below), provided that the indicated atom's normal valency is not exceeded, and that the substitution results in a stable compound. Suitable substituents of a substituted group can include alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acetylamino, nitro, trifluoromethyl, trifluoromethoxy, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl (alkyl)amine, and cyano. Additionally, the suitable indicated groups can include, e.g., —X, —R, —O—, —OR, —SR, —S—, —NR_{2,} —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O—, —S(═O)₂OH, —S(═O)₂R, —OS(═O)₂OR, —S(═O)₂NR, —S(═O)R, —OP(═O)O₂RR, —P(═O)O₂RR, —P(═O)(O—)₂, —P(═O)(OH)₂, —C(═O)R, —C(═O)X, —C(S)R, —C(O)OR, —C(O)O—, —C(S)OR, —C(O)SR, —C(S)SR, —C(O)NRR, —C(S)NRR, —C(NR)NRR, where each X is independently a halogen (“halo”): F, Cl, Br, or I; and each R is independently H, alkyl, aryl, heteroaryl, heterocycle, a protecting group or prodrug moiety. As would be readily understood by one skilled in the art, when a substituent is keto (═O) or thioxo (═S), or the like, then two hydrogen atoms on the substituted atom are replaced. Note that, in the final fluorophore, carbonyls and other chemical functionalities that render the molecule unstable against self-reaction are generally excluded, but the base fluorophore before modification or substitution may include such functionalities.

The terms “polynucleotides”, “nucleic acids”, “nucleotides”, “probes” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are nonlimiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. “Polynucleotide” may also be used to refer to peptide nucleic acids (PNA), locked nucleic acids (LNA), threofuranosyl nucleic acids (TNA) and other unnatural nucleic acids or nucleic acid mimics. Other base and backbone modifications known in the art are encompassed in this definition. See, e.g., De Mesmaeker et al (1997) Pure & Appl. Chem., 69, 3, pp 437-440.

The terms “polypeptide”, “peptide” and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfonation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “antibody” as used herein refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibody. These fragments are collectively termed “antigen-binding units”. Antigen binding units can be broadly divided into “single-chain” (“Sc”) and “non-single-chain” (“Nsc”) types based on their molecular structures.

The phrase “antibody mimetic” includes, without limitation, Affibody molecules, Affilins; Ubiquitin; Affimers; Affitins; Alphabodies; Anticalins; Avimers; DARPins; Fynomers; Kunitz domain peptides; Monobodies; and nanoCLAMPs. See, en.wikipedia.org/wiki/Antibody_mimetic.

Also encompassed within the terms “antibodies” are immunoglobulin molecules of a variety of species origins including invertebrates and vertebrates. The term “human” as applies to an antibody or an antigen binding unit refers to an immunoglobulin molecule expressed by a human gene or fragment thereof. The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

The term “stable” refers to compositions and compounds which have sufficient chemical stability to survive isolation from a reaction mixture to a useful degree of purity for use in a desired application.

The terms “fluorescent group”, “fluorophore”, “dye” or “fluorescent group” refer interchangeably to molecules, groups or radicals which are fluorescent. The term “fluorescent” as applied to a molecule of compound is used to refer to the property of the compound of absorbing energy (such as UV, visible or IR radiation) and re-emitting at least a fraction of that energy as light over time. Fluorescent groups, compounds or fluorophores include, but are not limited to discrete compounds, molecules, proteins and macromolecular complexes. Fluorophores also include compounds that exhibit long-lived fluorescence decay such as lanthanide ions and lanthanide complexes with organic ligand sensitizers.

The fluorescence “quantum yield” (φ) is the ratio of the number of photons emitted to the number absorbed. The term fluorescence quantum yield (φ) is a measure of the efficiency of photon emission through fluorescence, which is the loss of energy by a substance that has absorbed light via emission of a photon. In other words, the fluorescence quantum yield gives the probability of the excited state being deactivated by fluorescence rather than by another, non-radiative mechanism such as internal conversion or vibrational relaxation (non-radiative loss of energy as heat to the surroundings). The fluorescence intensity (F) is proportional to the amount of light absorbed, F=φ(Io-I), where lₒ is the intensity of the incident light and l is the intensity transmitted. Because I/Iₒ=10^{-slc} (Beer-Lambert law), the amount of light absorbed is: εIo-I=Io(1-10^{-εlc}), resulting in F=φIo(1-10^{-elc}), where ε a is the molar extinction coefficient, l is the optical path length, and c is the concentration. When the concentration of the fluorescent compound is low, the fluorescence intensity is given by the equation: εF=2.3φIoεlc.

A “subject” as used herein refers to a biological entity containing expressed genetic materials. The biological entity is in various embodiments, a vertebrate. In some embodiment, the biological entity is a mammal. In other embodiments, the subject is a biological entity which comprises a human.

A “control” is an alternative subject or sample used in an experiment for comparison purposes. A control can be “positive” or “negative”. For example, where the purpose of the experiment is to detect a differentially expressed transcript or polypeptide in cell or tissue affected by a disease of concern, it is generally preferable to use a positive control (a subject or a sample from a subject, exhibiting such differential expression and syndromes characteristic of that disease), and a negative control (a subject or a sample from a subject lacking the differential expression and clinical syndrome of that disease.

“Fluorophore” refers to a small molecule or a part of a large molecule, that can be excited by light to emit fluorescence. In some embodiments, fluorophores efficiently produce fluorescence upon excitation with light which has a wavelength from about 200 nanometers to about 1000 nanometers, e.g., from about 450 nanometers to about 800 nanometers. The intensity and wavelength of the emitted radiation generally depend on both the fluorophore and the chemical environment of the fluorophore.

A fluorophore may be selected from Other non-limiting examples can be found in The Handbook: a Guide to Fluorescent Probes and Labeling Technologies (10th Edition, Molecular Probes, Eugene, Oreg., 2006), which are incorporated herein by reference.

Such fluorophores include Acridine orange, Acridine yellow, anthracene ring, Alexa Fluor fluorescent groups, allophycocyanin, ATTO fluorescent groups, BODIPY fluorescent groups, Auramine O, Benzanthrone, 9,10-Bis(phenylethynyl)anthracene, 5,12-Bis(phenylethynyl)naphthacene, Carboxyfluorescein diacetate, Calcein, Carboxyfluorescein, 1-Chloro-9,10-bis(phenylethynyl)anthracene, 2-Chloro-9,10-bis(phenylethynyl)anthracene, Coumarin, Cyanine, Cy2, Cy3, Cy3.5, Cy5, Cy5.5, Cy7, DyLight Fluor fluorescent groups, Edans, Eosin, Erythrosin, Fluorescein, 2′,7′-dichlorodihydrofluorescein, fluorescamine, FAM (carboxyfluorescein), HEX (hexachlorofluorescein), Hilyte Fluor fluorescent groups, JOE (6-carboxy-4′,5′-dichloro-2′,7′-dimethoxy-fluorescein), LDS 751, Oregon Green, Perylene, Phycobilin, phycocyanin, Phycoerythrin, Phycoerythrobilin, Pyrene, Rhodamine and Ruthenium(II) tris(bathophenanthroline disulfonate), ROX (Carboxy-X-rhodamine), TAMRA (carboxytetramethylrhodamine), TET (tetrachloro-fluorescein), Texas Red®, tetramethylrhodamine, and xanthines. These compounds and derivatives or radicals of these compounds may be used as fluorophores.

Other examples of fluorescent groups which may be used include but are not limited to 4-acetamido-4′-isothiocyanatostilbene-2,2′disulfonic acid, acridine and derivatives such as acridine and acridine isothiocyanate, 5-(2′-aminoethyl)aminonaphthalene-l-sulfonic acid (EDANS), 4-amino-N-[3-vinylsulfonyl)phenyl]naphthalimide-3,5 disulfonate (Lucifer Yellow VS), N-(4-anilino-l-naphthyl)maleimide, anthranilamide, BODIPY™ and its derivatives and analogs, Brilliant Yellow, cyanine fluorescent groups such as Cy3 and Cy5 and other derivatives, coumarin and derivatives such as coumarin, 7-amino-4-methylcoumarin (AMC, Coumarin 120), 7-amino-4-trifluoromethylcouluarin (Coumaran 151), 7-diethylamino-3-(4′-isothiocyanatophenyl)-4-methylcoumarin, 5-[dimethylamino]naphthalene-1-sulfonyl chloride (DNS, dansyl chloride), fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; 4-methylumbelliferone, oxazine fluorescent groups such as Nile Blue and other analogs; pyrene and derivatives such as pyrene, pyrene butyrate and succinimidyl 1-pyrene butyrate; rosamine fluorescent groups, tetramethyl rosamine, and other analogs, rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA), tetramethyl rhodamine; tetramethyl rhodamine isothiocyanate (TRITC) and thiazine fluorescent groups such as methylene blue and analogs. Additional fluorophores applicable for use in the present are disclosed in US patent application Nos. 2003/0165942, 2003/0045717, and 2004/0260093 and U.S. Pat. No. 5,866,366 and WO 01/16375, both of which are incorporated herein by reference. Additional examples are described in U.S. Pat. No. 6,399,335, published U.S. patent application No. 2003/0124576, and The Handbook—‘A Guide to Fluorescent Probes and Labeling Technologies, Tenth Edition’ (2005) (available from Invitrogen, Inc./Molecular Probes), all of which are incorporated herein by reference.

Many such fluorescent groups are commercially available and may be used in the synthesis of compounds of the present invention. Commercial sources of reactive fluorescent groups include Invitrogen (Molecular Probes), AnaSpec, Amersham (AP Biotech), Atto-Tec, Dyomics, Clontech and Sigma-Aldrich.

For example, the fluorophore moiety is selected from the group consisting of dansyl, 4-(Diethylamino)azobenzene-4′-sulfonyl, fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions, quantum Dye™, fluorescent energy transfer dyes, thiazole orange-ethidium heterodimer, the cyanine dyes Cy3, Cy3.5, Cy5, Cy5.5 and Cy7,3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine,

Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.18, CY5.18, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Of course, the selected fluorophore prior to reaction should be stable, and not have a chemically-reactive carbonyl, and should otherwise have or be compatible with addition of a hydrazide or hydrazine functionality.

The term “FRET” refers to Förster resonance energy transfer. In the present invention, FRET refers to energy transfer processes occurring between at least two fluorescent compounds, between a fluorescent compound and a non-fluorescent component or between a fluorescent component and a non-fluorescent component.

A “binding agent” is a molecule that exhibits binding selectivity towards a binding partner or a target molecule to which it binds. A binding agent may be a biomolecule such as a polypeptide such as an antibody or protein, polypeptide-based toxin, amino acid, nucleotide, polynucleotides including DNA and RNA, lipids, and carbohydrates, or a combination thereof. A binding agent may also be a hapten, drug, ion-complexing agent such as metal chelators, microparticles, synthetic or natural polymers, cells, viruses, or other fluorescent molecules including the dye molecule according to the invention.

A “targeting moiety” is the portion of the binding agent that binds to a binding partner. A targeting moiety may be, without limitation, a nucleotide sequence within a polynucleotide that selectively binds to another polynucleotide or polypeptide. Another nonlimiting example of a targeting moiety may be a polypeptide sequence within a larger polypeptide sequence which binds specifically to a polynucleotide sequence or a second polypeptide sequence. A targeting moiety may be a small molecule or structural motif which will bind to a protein receptor, another small molecule motif, or complexing agent, without limitation. The selective binding may be a specific binding event.

A “binding partner” is a molecule or particle which is bound by the targeting moiety. It can be a cell, virus, fragment of a cell, antibody, fragment of an antibody, peptide, protein, polynucleotide, antigen, small molecule, or a combination thereof. It may be bound selectively or specifically by the binding agent.

The term “signal to noise ratio” of fluorescence as referred to herein is the ratio of (fluorescent signal from a complex targeted by the fluorophore)/(total fluorescent signal).

“Degree of labeling” or “DOL” as used herein refers to the number of dye molecules which are attached per target molecule (including but not limited to polypeptide and polynucleotide). For example, a single dye molecule per a polypeptide such as an antibody represents a 1.0 degree of labeling (DOL). If more than one dye molecule, on average, reacts with and is crosslinked to a polypeptide such as an antibody, the degree of labeling is greater than 1 and may further be a number other than a whole integer. The higher the number of DOL, the greater extent of labeling.

“Intracellular” as used herein refers to the presence of a given molecule in a cell. An intracellular molecule can be present within the cytoplasm, attached to the inner cell membrane, on the surface of an organelle, or within an organelle of a cell.

“Substrate” or “solid substrate” when used in the context of a reaction surface refers to the material that certain interaction is assayed. For example, a substrate in this context can be a surface of an array or a surface of microwell. It may also be a solid such as a polymer which does not form a specific shape but has attachment points on its surface.

The terms “wavelength of maximum excitation” and “maximal fluorescence excitation wavelength” are used herein interchangeably. These terms refer to the maximum wavelength at which a fluorescent compound absorbs light energy which excites the dye to emit maximal fluorescence. The term “absorption maximal wavelength” as applied to a dye refers the wavelength of light energy at which the dye most effectively absorbs. A fluorescent dye has a “maximal fluorescence emission wavelength” which is the wavelength at which the dye most intensely fluoresces. When a single wavelength is referred to for any dye, it refers to the maximal wavelength of excitation, absorption, or emission, according to the context of the term, for example, an absorption wavelength refers to the wavelength at which the compound has maximal absorption, and an emission wavelength refers to the wavelength at which the dye most intensely fluoresces.

Compounds of the invention comprise at least one Rₓ which is a reactive group. A reactive group is a chemical moiety capable of reacting with a reaction partner on a substrate or substrate molecule to form a covalent bond. A compound of the invention can be used to label a carbonyl. “Reactive group” and “reaction partner” refers to carbonyls, e.g., ketones and aldehydes.

An ionic group typically requires a counter ion to balance its charge. For example, negatively charged —SO3⁻ or CO₂⁻ groups may necessitate cations to balance the negative charge.

Likewise, a positively charged ammonium may require an anion to maintain neutrality. In general, the nature of the counter ion is not critical as long as the counter ion does not lower the solubility of said fluorescent group. In some embodiments, the counter ion is H⁺, Li⁺, Na⁺, K⁺, an ammonium group, Cl⁻, Br⁻, −CO₂⁻, —SO₃⁻, etc. If additional fluorescent compounds are used, such fluorescent compounds may intrinsically possess a positive charge or negative charge. In such a case, the intrinsic charge may act as a counter ion. Alternatively, the intrinsic charge may require a counter ion for maintaining neutrality.

The present invention also provides kits comprising compounds of the invention for various assays as selectively described above. A kit of the invention may comprise one or more compounds of the invention and instructions instructing the use of said compound. For example, a kit may comprise one or more compounds of the invention for labeling a substrate, one or more buffers for the labeling reaction and product purification, a protocol for carrying out the procedure, optionally any additional reagents and optionally any reference standard. In another embodiment, a kit comprises one or more fluorophores one or more buffers, a protocol for the use of said conjugate(s), optionally any other reagents for an assay, and optionally any calibration standard(s). The kit may be provided in a package, e.g., a shipping or storage container having at least two components, such as two dyes, and/or instructions, within the same container.

The signals produced by the fluorescent groups of the invention may be detected in a variety of ways. Generally, a change of signal intensity can be detected by any methods known in the art and is generally dependent on the choice of fluorescent group used. It can be performed with the aid of an optical system. Such system typically comprises at least two elements, namely an excitation source and a photon detector. Numerous examples of these elements are available in the art. An exemplary excitation source is a laser, such as a polarized laser. The choice of laser light will depend on the fluorescent group attached to the probe. For most of the fluorescent groups, the required excitation light is within the range of about 300 nm to about 1200 nm, or more commonly from about 350 nm to about 900 nm. Alternatively, compounds of the invention may be excited using an excitation wavelength of about 200 to about 350 nm, 350 to 400 nm, 400 to 450 nm, 450 to 500 nm, merely by way of example. Those skilled in the art can readily ascertain the appropriate excitation wavelength to excite a given fluorophore by routine experimentation, (see e.g., The Handbook—“A Guide to Fluorescent Probes and Labeling Technologies, Tenth Edition” (2005) (available from Invitrogen, Inc./Molecular Probes) previously incorporated herein by reference). Where desired, one can employ other optical systems. These optical systems may comprise elements such as optical reader, high-efficiency photon detection system, photo multiplier tube, gate sensitive FET's, nano-tube FET's, photodiode (e.g., avalanche photo diodes (APD)), camera, charge couple device (CCD), electron-multiplying charge-coupled device (EMCCD), intensified charge coupled device (ICCD), and confocal microscope. These optical systems may also comprise optical transmission elements such as optic fibers, optical switches, mirrors, lenses (including microlens and nanolens), collimators. Other examples include optical attenuators, polarization filters (e.g., dichroic filter), wavelength filters (low-pass, band-pass, or high-pass), wave-plates, and delay lines. In some embodiments, the optical transmission element can be planar waveguides in optical communication with the arrayed optical confinements. See, e.g., U.S. Pat. Nos. 7,292,742, 7,181,122, 7,013,054, 6,917,726, 7,267,673, and 7,170,050. These and other optical components known in the art can be combined and assembled in a variety of ways to effect detection of distinguishable signals.

Fluorescently labeled products of the invention find use in a variety of applications. Such applications can involve interactions between nucleic acids, e.g., interactions between DNA and DNA, DNA and RNA, and RNA and RNA, or any other non-naturally occurring nucleic acids PNA, LNA, and/or TNA. Various applications can also involve interactions between nucleic acids and proteins, lipids or combinations thereof. Non-limiting examples of specific nucleic acid assays include nucleic acid amplification, both quantitative or end-point amplification, hybridization in solution or on a substrate (e.g., array hybridization), gel shifts, and nucleic acid sequencing. The fluorescently labeled polynucleotides can be used in solution phase or immobilized on a substrate. By labelling based on the somewhat random location of carbonyl formation, the fluorescently labelled products will be a family of compounds, and therefore location-dependent of reaction will tend to be diluted.

According to one embodiment of the invention, biomolecules labeled with a fluorescent group of the invention such as proteins are suitable for in vivo imaging, including without limitation imaging a biomolecule present inside a cell, a cell, tissue, organ or a whole subject. Where desired, the labeled biomolecules can be used to perform “In Cell Western” in which given molecules (e.g., a specific cellular protein) present inside a cell are stained and imaged.

The compounds of the invention or the labeled biomolecules of the invention can also be used to label cells or particles for a variety of applications. Accordingly, the present invention provides a method of individually labeling a cell within a population of cells whereby the cell is differentially labeled relative to neighboring cells within the population. The method typically comprises contacting the cell with a labeled biomolecule of the present invention, wherein said biomolecule comprises a targeting moiety that binds to a binding partner that is indicative of said cell, and thereby differentially labeling the cell relative to neighboring cells within the population. The targeting moiety can be any biomolecules that recognize a binding partner on the cell to be detected. The choice of the targeting moiety will vary depending on the cell that is to be labeled. For example, for detecting a cancer cell, a targeting moiety is selected such that its binding partner is differentially expressed on a cancer cell. A vast number of cancer markers are known in the art. They include without limitation cell surface receptors such as erb2, PDGF receptor, VEGF receptors, a host of intracellular proteins such as phosphatidylinositol 3-kinases, c-abl, raf, ras, as well as a host of nuclear proteins including transcription factors and other nucleic acid binding molecules. In some other embodiments, the cancer marker is

Immunoglobulin epsilon Fc receptor II, Alk-1, CD20, EGF receptor, FGF receptor, NGF receptor, EpCam, CD3, CD4, CD11 a, CD19, CD22, CD30, CD33, CD38, CD40, CD51, CD55, CD80, CD95, CCR2, CCR3, CCR4, CCR5, CTLA-4, Mucin 1, Mucin 16, Endoglin, Mesothelin receptor, Nogo receptor, folate receptor, CXCR4, insulin-like growth factor receptor, Ganglioside GD3, and alpha or beta Integrins. To differentially label various cell types, targeting moieties recognizing a cell-specific binding partner can be used. For example, there are a host of protein markers differentially expressed on T cells as opposed on B cells or other cells of different lineage. Neuronal markers, muscle cell markers, as well as markers indicative of cells of ectodermal, mesodermal or endodermal origins are also known in the art, all of which can be used depending on the intended applications. The targeting moieties can be antibodies, receptors, cytokines, growth factors, and any other moieties or combinations thereof that are recognized by a binding partner on the cell to be labeled. The cell which is labeled may be labeled intracellularly.

Note that cells, and in particular immune cells, respond to stimuli by a metabolic burst of activity, that will result in oxidative stress. Therefore, responding cell populations may be distinguished from non-responding cell populations, by use of fluorophores that selectively label the responding population. Detection of this gross level of response, especially in live cells, and wherein the assay may be conducted under non-lethal conditions, is useful in experiments where the underlying mechanisms and receptors are not yet elucidated.

The differentially labeled cells can be imaged by directing exciting wavelength to the cell and detecting emitted fluorescence from the cell, in a number of in-vitro formats, either in solution or immobilized on a substrate.

The labeled cells and/or the intensity of the fluorescence may be detected or quantified by performing flow cytometry. Cells or particles labeled with the compounds of the invention or stained with labeled biomolecules of the invention may also be separated and isolated based on the specific properties of the label using fluorescence activated cell sorting (FACS). Such techniques are known in the art. Briefly, cells are labeled with a subject fluorescent dye and then passed, in a suspending medium, through a narrow dropping nozzle so that each cell is typically in a small droplet. A laser-based detector system is used to excite fluorescence and droplets with positively fluorescent cells are given an electric charge. Charged and uncharged droplets are separated as they fall between charged plates and so collect in different tubes. The machine can be used either as an analytical tool, counting the number of labeled cells in a population or to separate the cells for subsequent growth of the selected population. Further sophistication can be built into the system by using a second laser system at right angles to the first to look at a second fluorescent label or to gauge cell size on the basis of light scatter.

Additional guidance for performing fluorescent cell sorting can be found in publications such as the following: Darzynkiewicz, Z., Crissman, H. A. and Robinson, J. P., Eds., Cytometry, Third Edition Parts A and B (Methods in Cell Biology, Volumes 63 and 64), Academic Press (2001); Davey, H. M. and Kell, D. B., “Flow cytometry and cell sorting of heterogeneous microbial populations: the importance of single-cell analyses,” Microbiological Rev 60, 641-696 (1996); Givan, A. L., Flow Cytometry: First Principles, Second Edition, John Wiley and Sons (2001); Herzenberg, L. A., Parks, D., Sahaf, B., Perez, O., Roederer, M. and Herzenberg, L. A., “The history and future of the fluorescence activated cell sorter and flow cytometry: a view from Stanford,” Clin Chem 48, 1819-1827 (2002); Jaroszeski, M. J. and Heller, R., Eds., Flow Cytometry Protocols (Methods in Molecular Biology, Volume 91), Humana Press (1997); Ormerod, M. G., Ed., Flow Cytometry: A Practical Approach, Third Edition, Oxford University Press (2000); Robinson, J. P., Ed., Current Protocols in Cytometry, John Wiley and Sons (1997); Shapiro, H. M., “Optical measurement in cytometry: light scattering, extinction, absorption and fluorescence,” Meth Cell Biol 63, 107-129 (2001); Shapiro, H. M., Practical Flow Cytometry, Fourth Edition, Wiley-Liss (2003); Weaver, J. L., “Introduction to flow cytometry,” Methods 21, 199-201 (2000).

Fluorescent compounds of the invention may also be used for fluorescence lifetime imaging (FLIM). FLIM is a useful technique for producing images based on the variation in the fluorescence decay characteristics of a fluorescent sample. It can be used as an imaging technique in confocal microscopy and other microscope systems. The lifetime of the fluorophore signal, rather than its intensity, is used to create the image in FLIM, which has the advantage of minimizing the effect of photon scattering in thick layers of sample. FLIM may be useful for biomedical tissue imaging, allowing to probe greater tissue depths than conventional fluorescence microscopy.

The present compounds may also be used for the labeling of lipids. Lipids are involved in many biological processes, and the labeling of lipids and lipid rafts may is often a valuable method for studying their properties. Various lipid monolayers and bilayers may be labeled in live cells or artificial systems such as liposomes and micelles. For example, a live cell population may be labeled with a fluorescent conjugate prepared by reacting a compound of the invention and cholera toxin subunit B, which specifically interacts with lipid rafts. Such lipid rafts may then be crosslinked into distinct membrane patches by the use of an anti-cholera toxin antibody, which may be labeled with one of the present compounds.

The labeled polypeptides of the present invention find use as biosensors in prokaryotic and eukaryotic cells.

The subject fluorescent proteins also find use in applications involving the automated screening of arrays of cells by using microscopic imaging and electronic analysis. Screening can be used for drug discovery and disease diagnosis.

It is an object to provide a method of detecting carbonyls in living cells, comprising:

incubating the living cells with a fluorophore having at least one of a hydrazine group, an alkoxyamine group, and a hydrazide group; reacting the fluorophore with carbonylated biomolecules of the living cells to couple the fluorophore to the biomolecules through a hydrazone or oxime linkage; and detecting the fluorophore by imaging the cells.

It is another object to provide a method of detecting carbonylated lipids in living cells, comprising incubating the living cells having the carbonylated lipids with a fluorophore having at least one of a hydrazine group, an alkoxyamine group, and a hydrazide group; reacting the fluorophore with carbonylated lipids to couple the fluorophore to the biomolecules through a hydrazone or oxime linkage; and detecting the carbonylated lipids by imaging.

It is a further object to provide a method of detecting carbonylated nucleic acids in living cells, comprising incubating the living cells having the carbonylated nucleic acids with a fluorophore having at least one of a hydrazine group, an alkoxyamine group, and a hydrazide group; reacting the fluorophore with carbonylated nucleic acids to couple the fluorophore to the biomolecules through a hydrazone or oxime linkage; and detecting the carbonylated nucleic acids by imaging.

Another object provides a method of detecting carbonylated carbohydrates in living cells, comprising incubating the living cells having the carbonylated carbohydrates with a fluorophore having at least one of a hydrazine group, an alkoxyamine group, and a hydrazide group; reacting the fluorophore with carbonylated carbohydrates to couple the fluorophore to the biomolecules through a hydrazone or oxime linkage; and detecting the carbonylated carbohydrates by imaging.

It is a still further object to provide a reaction product of 7-hydrazinyl-4-trifluoromethylcoumarin (TFCH), or a 7-hydrazinyl coumarin derivative having at least one fluoromethyl substituent, or a 7-hydrazinyl coumarin derivative having a trifluoromethyl substituent, or a lipohilic 7-hydrazinyl coumarin derivative having a 4-nucleophilic substituent and at least one of a carbonylated lipid, a carbonylated nucleic acid, a carbonylated carbohydrate, a carbonylated polypeptide.

The fluorophoes according to the present invention may be used to distinguish between cancer cells and normal cells; assess cell aging and senescence; and assess a toxic insult to the cells, based on a level of carbonylation of biomolecules in the living cells.

TFCH is believed to have a quantum yield of >0.1 in water. Note that fluorescent quantum yield of a fluorphor in water is typically lower than in other solvents. Bindhu, C. V., S. S. Harilal, V. P. N. Nampoori, and C. P. G. Vallabhan. “Solvent effect on absolute fluorescence quantum yield of rhodamine 6G determined using transient thermal lens technique.” Modern physics letters B 13, no. 16 (1999): 563-576; Lee, J., and H. H. Seliger. “Quantum yields of the luminol chemiluminescence reaction in aqueous and aprotic solvents.” Photochemistry and Photobiology 15, no. 2 (1972): 227-237; Kokubun, Hiroshi. “Fluorescence lifetime of acridine in water-organic solvent mixtures.” Bulletin of the Chemical Society of Japan 42, no. 4 (1969): 919-922.

Another object provides a method of detecting carbonylated macromolecules in living cells, comprising incubating the living cells having the carbonylated macromolecules with a 7-hydrazinyl coumarin fluorophore having an optional 3-substituent or 4-substituent; reacting the fluorophore with the carbonylated macromolecules in the living cells to form a hydrazone or oxime linkage; and detecting the fluorophore linked through the hydrazone or oxime linkage to the macromolecules by imaging.

A further method provides a method of assessing reactive oxygen species formation in living cells, comprising subjecting the living cells to a treatment which causes varying amounts of reactive oxygen species formation, to form carbonyls on biomolecules; incubating the living cells having the carbonyls on the biomolecules with a fluorophore having at least one of a hydrazine group, an alkoxyamine group, and a hydrazide group; reacting the fluorophore with the carbonyls on the biomolecules of the living cells to react the at least one of a hydrazine group, an alkoxyamine group, and a hydrazide group with the carbonyls to form a hydrazone or oxime linkage; and quantitatively assessing the fluorophore by fluorescent imaging.

A still further object provides a method of assessing carbonylated biomolecules, comprising fixing the cells, e.g., with methanol or other fixative, while preserving lipid membranes; reacting the fixed cells in serum free media with a fluorophore having at least one of a hydrazine group, an alkoxyamine group, and a hydrazide group, to form a hydrazone or oxime linkage of the fluorophore with carbonylated biomolecules of the fixed cells; assessing a spatial distribution of the fluorophore by fluorescent imaging. The spatial distribution may comprise the preserved lipid membranes.

Another object provides a method of detecting carbonylated molecules in living cells, comprising adding a fluorophore having at least one of a hydrazine, alkoxyamine, or hydrazide substituent which is cell membrane permeable and reactive with the carbonylated molecules to the living cells having the carbonylated molecules, to form a hydrazone or oxime linking the fluorophore with the molecules; and optically inspecting the cells to visualize portions of the cells to which the fluorophore is bound.

The fluorophore having at least one of a hydrazine group, an alkoxyamine group, and a hydrazide group may comprise 7-hydrazinyl-4-trifluoromethylcoumarin (TFCH).

The fluorophore may have a fluorescent emission peak which changes as a result of the hydrazone or oxime formation.

The method may further comprise washing excess fluorophore from the cells.

The linkage may be a hydrazone. The linkage may be an oxime.

The fluorophore may have a hydrazine group. The fluorophore may have a hydrazide group. The fluorophore may have an alkoxyamine group. The fluorophore may be a coumarin derivative, fluorescein derivative, a rhodamine derivative, or a BODIPHY derivative.

The carbonylated biomolecules may be proteins, nucleic acids, lipids, and/or carbohydrates.

The living cells may be in a bodily fluid, which may be, e.g., blood, urine, cerebrospinal fluid, semen, blood plasma, saliva, synovial fluid, serum, mucus, tears, amniotic fluid, lymph, breast milk, vaginal lubrication, bile, aqueous humour, hemoglobin, gastric acid, phlegm, vitreous body, transudate, pericardial effusion, interstitial fluid, pus, ground substance, earwax, rheum, colostrum, chyle, intestinal juice, human feces, glycocalyx, or chyme.

The imaged cells may be alive or may be preserved (e.g., fixed) prior to imaging. The optical inspection may be of the live or preserved (e.g., fixed) cells. The fixative may be methanol.

The cells may be imaged using fluorescence microscopy, confocal microscopy, or fluorescent confocal microscopy.

The living cells may be in cell culture. The living cells may be in a biopsy specimen. The living cells may be in blood.

The optical inspection may be of living or fixed cells. The cells may be fixed with methanol, or other fixative.

The optically inspecting may be prior to, concurrent with, or subsequent to optically inspecting the cells to visualize portions of the cells to which at least one other distinct fluorophore is bound. The optically inspecting may be part of a multiplex assay. The optically inspecting may be part of a high throughput screening assay. The optically inspecting may be performed using optical flow cytometry.

The at least one other distinct fluorophore may be Resazurin, sulforhodamine B, pyridium iodide, DRAQ5, DRAQ7, resazurin, ROS-Glo™ H₂O₂ CellROX® Deep Red, CellROX® Green and CellROX® Orange; CM-H2-DCFDA; Image-It, MitoSOX Red Superoxide Indicator; Invitrogen Premo Cellular Hydrogen Peroxide H2O2 Sensor; Invitrogen Premo Cellular Redox Sensor, Grx-1-roGFP; ThiolTracker Violet dye; NucGreen Dead 488 ReadyProbes Reagent, NucRed Dead 647 ReadyProbes Reagent, ReadyProbes Cell Viability Imaging Kit (Blue/Green), or ReadyProbes Cell Viability Imaging Kit (Blue/Red).

At least one drug or therapy may be administered drug to the living cells prior to adding the fluorophore. The living cells may be in a multiwell plate, with different treatments and/or replicates of the same treatment in different wells. The living cells may thus be provided in portions of cell culture aliquots in different wells of a multi-well plate, further comprising administering at least two distinct treatments to the living cells of different wells prior to adding the fluorophore.

A further object provides a reagent, comprising 7-hydrazinyl-4-trifluoromethylcoumarin (TFCH) hydrochloride or 7-hydrazinyl-4-trifluoromethylcoumarin (TFCH) and a buffer salt or 7-hydrazinyl-4-trifluoromethylcoumarin (TFCH) and an anionic counterion or a biocompatible salt of 7-hydrazinyl-4-trifluoromethylcoumarin (TFCH) with a buffer within a range of pH 6 to pH 8.5. The reagent may be provided as a dry solid, e.g., in a glass vial, preferably a colored or brown glass vial. The contents of the vial may be reconstituted with a predetermined volume of liquid medium, such as DMSO or a buffer, to produce a reconstituted solution. The vial may comprise between 200 μg and 2 mg TFCH, e.g., 1 mg TFCH.

It is another object to provide a method of detecting carbonylated biomolecules in living cells, comprising incubating the living cells with a fluorophore having at least one of a hydrazine group, an alkoxyamine group, and a hydrazide group; reacting the fluorophore with carbonylated biomolecules of the living cells to couple the fluorophore to the biomolecules through a hydrazone or oxime linkage; and detecting the fluorophore by imaging under illumination of the fluorophore reacted with the carbonylated biomolecules, having a limit of detection (LOD) in cell lysate of less than 100 nM. The method optionally includes a washing step before imaging to remove unbound fluorophore, or the imaging may be performed on an unwashed specimen. The cells, when images, may be living or fixed. The fluorophore having at least one of a hydrazine group or a hydrazide group may comprises 7-hydrazinyl-4-trifluoromethylcoumarin (TFCH), and the fluorophore reacted with the carbonylated biomolecules may comprises 7-hydrazinyl-4-trifluoromethylcoumarin hydrazone (TFCZ).

The fluorophore preferably has a fluorescent emission peak which shifts as a result of the hydrazone or oxime formation, to distinguish the fluorophore and the reaction product during the detection. The imaging may be 2-photon microscopy.

The fluorophore coupled to the biomolecules through the hydrazone or oxime linkage may comprises a 7-hydrazinyl, 7-alkoxyamine or 7-hydrazide coumarin derivative having a 4-nucleophilic substituent, having a quantum yield of greater than 0.1 in 0.5% DMSO in phosphate buffered water at pH 7.0 or pure water.

It is another object to provide a method of detecting carbonylated macromolecules in living cells, comprising incubating the living cells having the carbonylated macromolecules with a fluorophore comprising a 7-hydrazinyl, 7-alkoxyamine or 7-hydrazide coumarin derivative having a 4-nucleophilic substituent to form a fluorophore conjugated to the carbonylated macromolecules through a hydrazone or oxime linkage; illuminating the conjugated fluorophore at a first wavelength; and detecting the conjugated fluorophore by imaging fluorescent emissions at a second wavelength within 90 minutes of commencement of the incubating, wherein fluorescent emissions of the fluorophore at the second wavelength are substantially non-interfering. The conjugated fluorophore preferably has a quantum yield of greater than 0.1 in 0.5% DMSO in phosphate buffered water at pH 7.0. The method may further comprise exposing the living cells to a drug prior to incubating, wherein the carbonylated macromolecules result from reactive oxygen species caused by metabolism of the drug by the living cells, and the detecting comprises quantifying free radical-induced damage to the living cells from the drug.

The fluorophore may comprise 7-hydrazinyl 4-trifluoromethyl coumarin, and may be pure or essentially pure 7-hydrazinyl 4-trifluoromethyl coumarin. In one embodiment, a multiplex assay is performed, and the detecting is concurrent with fluorescent imaging of a distinct fluorophore at a different wavelength from the second wavelength. The distinct fluorophore may be selected from the group consisting of resazurin, sulforhodamine B, and pyridium iodide, providium iodide, and a 1,5-bis{[2-(di-methylamino) ethyl]amino}-4,8-dihydroxyanthracene-9,10-dione.

The incubating may comprise providing a multiwell plate having a plurality of wells, each well having living cells in a medium, and adding a different drug treatment to different subsets of the plurality of wells.

It is a further object to provide a reaction product of a fluorophore comprising a 7-hydrazinyl, 7-alkoxyamine, or 7-hydrazide, 4-nucleophilic substituted, coumarin, and at least one of a carbonylated lipid, a carbonylated nucleic acid, a carbonylated carbohydrate, and a carbonylated polypeptide. The 7-hydrazinyl 4-nucleophilic substituted coumarin derivative may be 7-hydrazinyl-4-trifluoromethylcoumarin, 7-alkoxyamine 4-trifluoromethylcoumarin, or 7-hydrazide 4-trifluoromethylcoumarin. Thre fluorphore may have a 3-substituent other than hydrogen. The fluorophore may have a quantum yield of greater than 0.1 in 0.5% DMSO in phosphate buffered water at pH 7.0 and a limit of detection of less than 500 nM, 400 nM, 300 nM, 250 nM, 200 nm, 150 nM, 125 nM, 120 nM, 115 nM, 110 nM, 105 nM, 100 nM, 95 nM, 90 nM, and for 7-hydrazinyl-4-trifluoromethylcoumarin, is about 89 nM.

### BRIEF DESCRIPTION OF THE DRAWINGS

FIG. 1 shows tools and techniques for detection of biomolecule carbonylation in cell lysate (FIG. 1A) and intact cells (FIG. 1B).

FIG. 2 shows a schematic representation of recommended assay for detection of carbonylation in live cells.

FIG. 3 shows a schematic representation of one-step assay for carbonylation detection in live cells.

FIG. 4 shows a schematic representation of multiplex assay for detection of carbonylation and ROS production in live cells.

FIG. 5 shows results of menadione-induced carbonylation as detected in live cells.

FIG. 6 shows results of serum starvation-induced carbonylation as detected in live cells by a one-step assay.

FIGS. 7A and 7B show results of a multiplex assay for carbonyls and ROS induced by serum starvation in live cells.

FIG. 8A shows a schematic representation of hydrazone formation. FIGS. 8B-8D show structures of commercially available hydrazide fluorophores that were investigated: (FIG. 8B) DCCH, (FIG. 8C) BODIPYH, (FIG. 8D) TxRH.

FIGS. 9A-9C show a set of graphs for quantitative analysis of carbonyls in live cells in a high throughput platform.

FIGS. 10A-10C show a set of graphs for quantitative analysis of carbonyls in cell lysate.

FIG. 11 shows a set of fluorescent confocal microscopy images for detection and visualization of carbonyls in live cells.

FIG. 12 shows a set of fluorescent confocal microscopy images for detection and visualization of carbonyls in fixed A549 cells.

FIG. 13 shows a set of fluorescent confocal microscopy images for detection and visualization of carbonyls in fixed Hs 578B st (normal) and Hs 578T (carcinoma) cells.

FIGS. 14A and 14B show flow charts for cellular carbonyl detection assays. FIG. 14A shows an endpoint or fixed cell assays. FIG. 14B shows real-time or live cell assays.

FIGS. 15A-15F show absorption (top) and emission (bottom) spectra of (FIGS. 15A, 15B) DCCH, (FIGS. 15C, 15D) BODIPYH, and (FIGS. 15E, 15F) TxRH and their corresponding hydrazones with propanal.

FIGS. 16A-16C shows a set of graphs for hydrazide fluorophore efflux from A549 cells.

FIG. 17 shows a time course of hydrazone formation in live A549 cells.

FIGS. 18A-18C show optimization of assay conditions for detecting and visualizing carbonyls in fixed cells. FIG. 18A shows a set of fluorescent confocal microscopy images for PBS and serum free media. FIG. 18B shows DCCH and FIG. 18C shows TxRH.

FIG. 19A-19F show TFCH, a fluorescent probe for oxidative-stress induced carbonylation in live cells.

FIGS. 20A-20E show photochemical properties of TFCH and TFCZ.

FIGS. 21A-21E show that TFCH is compatible with live-cells, for detecting oxidative stress-induced carbonylation.

FIGS. 22A-22C show that TFCH serves as a tool for detecting oxidative stress-induced carbonylation in live cells by HCS- and HTS-compatible assays.

FIG. 23 shows a schematic representation of an automated fluorescence microscope screening system.

### DETAILED DESCRIPTION OF THE PREFERRED EMBODIMENTS

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

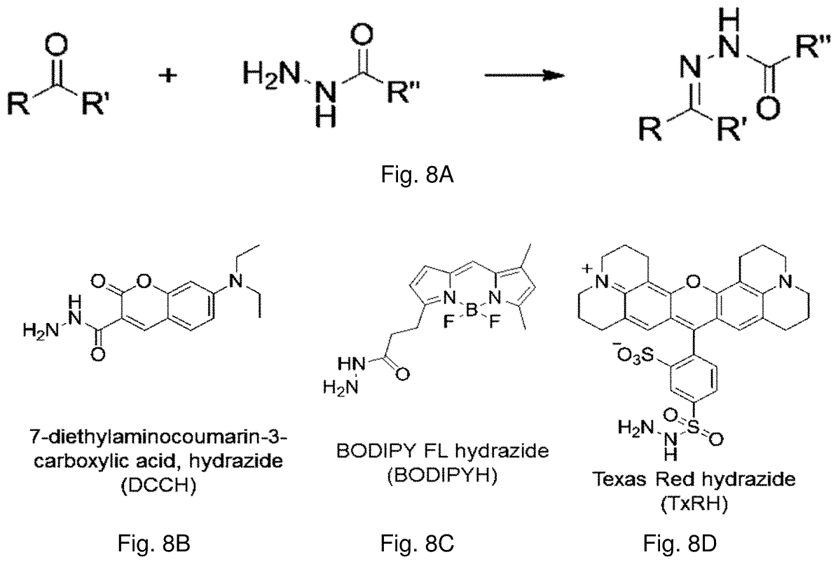

Fluorophores e.g., 7-Diethylaminocoumarin-3-carboxylic acid, hydrazide (DCCH, Millipore Sigma), BODIPYTM FL hydrazide, BODIPYH (ThermoFisher), Texas Red™ hydrazide, TxRH (ThermoFisher) or 7-Hydrazinyl-4-trifluoromethylcoumarin (TFCH, synthetic) are solubilized in DMSO (Acros), and diluted with DMSO to a final concentration of 2 mM, snap frozen in 6-10 μL aliquots (sufficient for single use) and stored at ≤−20° C. Snap frozen aliquots of TFCH should be used within one week of preparation. FIG. 8A shows a schematic representation of hydrazone formation. FIGS. 8B-8D show structures of commercially available hydrazide fluorophores that were investigated: (FIG. 8B) DCCH, (FIG. 8C) BODIPYH, (FIG. 8D) TxRH.

Of these fluorophores, TFCH is unexpectedly superior, in that the images are brighter, i.e., with a limit of detection about 10-fold lower that BzCH. The TFCH is also more photostable than BzCH. The TFCH has a higher absorption at 405 nm than the BzCH.

Synthesis and Characterization of TFCH and TFCZ

4-Trifluoromethyl-7-hydrazinyl-2H-chromen-2-one (TFCH)

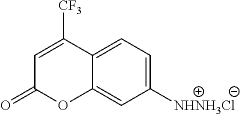

7-Amino-4-(trifluoromethyl) coumarin (500 mg, 2.18 mmol) was dissolved in 1.50 mL concentrated HCl and stirred for 10 min at −10° C. A chilled solution of sodium nitrite (181 mg, 1.2 eq) in 600 μL water was added dropwise to keep the temperature of reaction below 0° C. The solution was stirred for 1 h at −10° C. Stannous chloride dihydrate (1.57 mg, 3.8 eq) was dissolved in 1.50 mL concentrated HCl and chilled on ice. This cold stannous chloride HCl solution was then slowly added to the diazonium solution and the temperature of reaction was maintained below 0 ° C. The reaction was stirred for 1.5 h at −10° C. The yellow slurry was then filtered and washed with cold water and cold ethanol. TFCH HCl salt was collected as a light yellow solid (350 mg, 57% yield).

¹H NMR (400 MHz, DMSO-d6) δ: 10.49 (br, NH, 3H), 9.32 (s, NH, 1H), 7.59 (d, J=8.0 Hz, 1H), 7.00 (m, 2H), 6.77 (s, 1H).

¹³C NMR (400 MHz, DMSO-d6) δ: 159.25, 155.98, 151.00, 126.06, 123.59, 120.86, 112.53 (q, CF₃), 111.87, 105.97, 100.31.

MS-ESI^{+l : C}₁₀H₇F₃N₂O₂[M+H]⁺ calcd.: 245.17, found: 245.12.

7-(2-Propylidenehydrazinyl)-4-(trifluoromethyl)-2H-chromen-2-one (TFCZ)

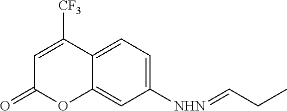

The TFCH HCl salt (20 mg, 0.082 mmol) was dissolved in 600 μL methanol and 20 μL of trifluoroacetic acid. Propionaldehyde (58.7 μL, 10 eq) was added to the solution which was stirred further for 30 min. The yellow precipitate was filtered and washed with cold methanol. The TFCZ was dried by air and collected as yellow solid (15 mg 64% yield).

¹H NMR (400 MHz, DMSO-d6) δ: 10.66 (s, NH, 1H), 7.50 (dd, J=9.0, 2.1 Hz, 1H), 7.37 (t, J=4.9 Hz, 1H), 6.94 (dd, J=9.0, 1.7 Hz, 1H), 6.86 (d, J=2.0 Hz, 1H), 6.57 (s, 1H), 2.30 (dq, J=7.5, 5.0 Hz, 2H), 1.08 (t, J=7.4 Hz, 3H).

¹³C NMR (400 MHz, DMSO-d6) δ: 159.65, 156.80, 150.37, 147.22, 126.43, 123.75, 121.01, 110.27, 109.71 (q, CF3), 104.11, 97.76, 25.76, 11.21.

MS-ESI⁺: C₁₃H₁₁F₃N₂O₂[M+H]⁺ calcd.: 285.08, found: 285.11.The following equation from Beer-Lambert Law is used to determine fluorophore concentration A_{λ}=ε_{λ} c l , where:

A_{λ}: Absorbance of the fluorophore solution at wavelength X,

ε_{λ}: Extinction coefficient of the chemical species in solution at wavelength 2\,

l: Path length or light path (usually 1 cm or corrected to 1 cm by instrument)

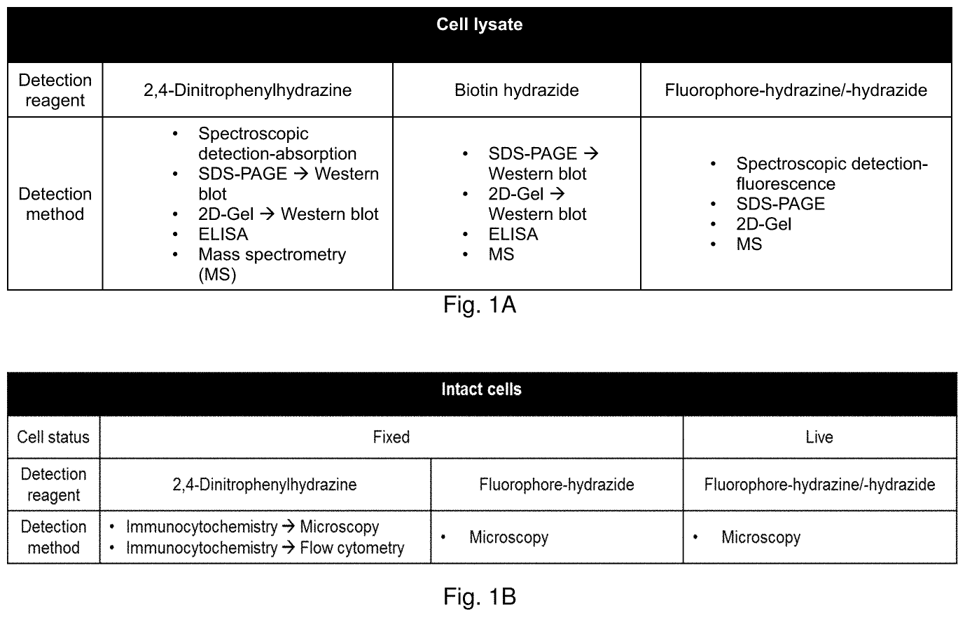

FIGS. 1A and 1B show tools and techniques for detection of biomolecule carbonylation in cell lysate (FIG. 1A) and intact cells (FIG. 1B).

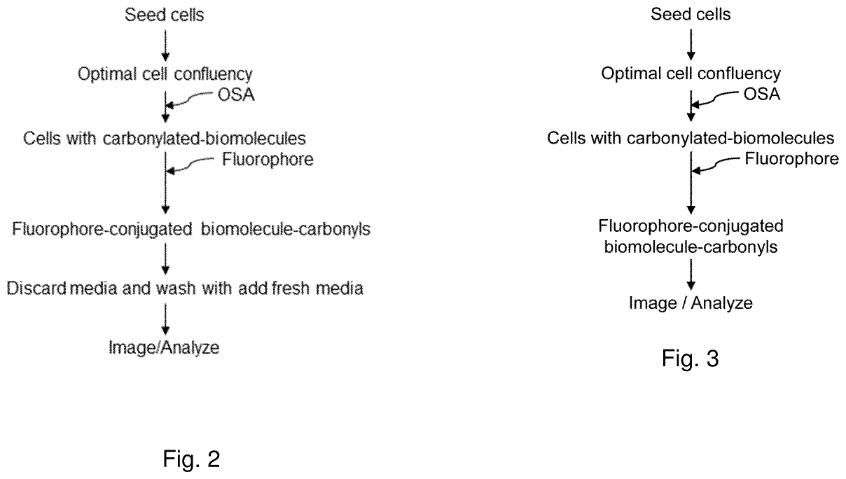

FIG. 2 shows a schematic representation of recommended assay for detection of carbonylation in live cells.

Seed cells in 200-400 μL medium (Dulbecco's phosphate buffer saline, DPBS (Millipore Sigma)) per well in an 8-well chambered coverglass (ThermoFisher). A cell number is used that will result in 50-90% confluency the following day. Cell confluency should be decided based on: OSA (oxidative stress-inducing agent) exposure time; Cytotoxicity associated with the OSA; and Doubling time of the cell line. For example, for 100 μM menadione exposure for 1 h, 60-80% confluent A549 cells are used.

Once the cells are at the desired confluency, the medium in the wells is discarded, and 200 μL fresh medium supplemented with the OSA or vehicle is added, and the samples returned to the cell culture incubator.

At the end of the incubation period with the OSA or vehicle, 1 μL of 2 mM fluorophore (final concentrations: fluorophore=10 μM; DMSO=0.5%, v/v) is added to one corner of each well, taking care not to touch the bottom of the well with the pipet tip, and mixed well, by gently moving the chambered coverglass in a circular motion (clockwise as well as counter-clockwise). The coverglass is then returned to the cell culture incubator and incubated for 30-45 min. The difference in fluorescence between the control and the sample may be affected by the time of incubation with the fluorophore and the cell line. The incubation time is adjusted based on the cell line and oxidant used. The medium is discarded and 500 μL fresh medium added to each well. The coverglass is returned to the cell culture incubator for 3 min. The washing step is repeated two more times. The final wash medium is discarded, and 200 μL of fresh medium added, and the cells imaged by, e.g., confocal microscopy.

If significant cell detachment is observed due to the severity of the OSA, one of the following could be done: washing the cells only one time with excess medium before imaging without a final wash, and performing a one-step detection assay, as described below.

FIG. 3 shows a schematic representation of one-step assay for carbonylation detection in live cells.

A one-step detection method for oxidative stress-induced carbonylation in live cells is similar to described above. The 2 mM stock solution of TFCH is diluted with DMSO to 400 μM. At the end of the incubation period with the OSA, 1 μL of 400 μM TFCH is added (Final concentrations: fluorophore=2 μM; DMSO=0.5%, v/v). The fluorophore solution is added to one corner of each well, taking care not to touch the bottom of the well with the pipet tip, and mixed well. The contents of the well are mixed by gently moving the chambered coverglass in a circular motion (clockwise as well as counter-clockwise). The coverglass is returned to the cell culture incubator and incubated for 30 min.

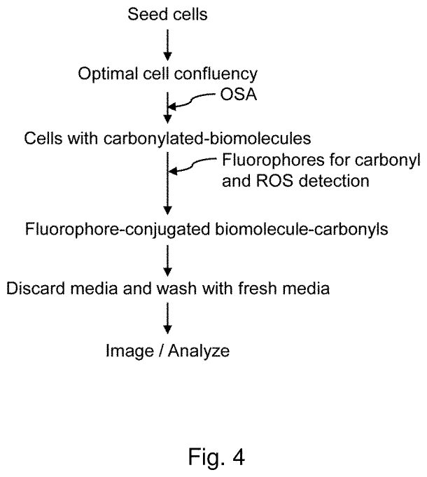

FIG. 4 shows a schematic representation of multiplex assay for detection of carbonylation and ROS production in live cells.

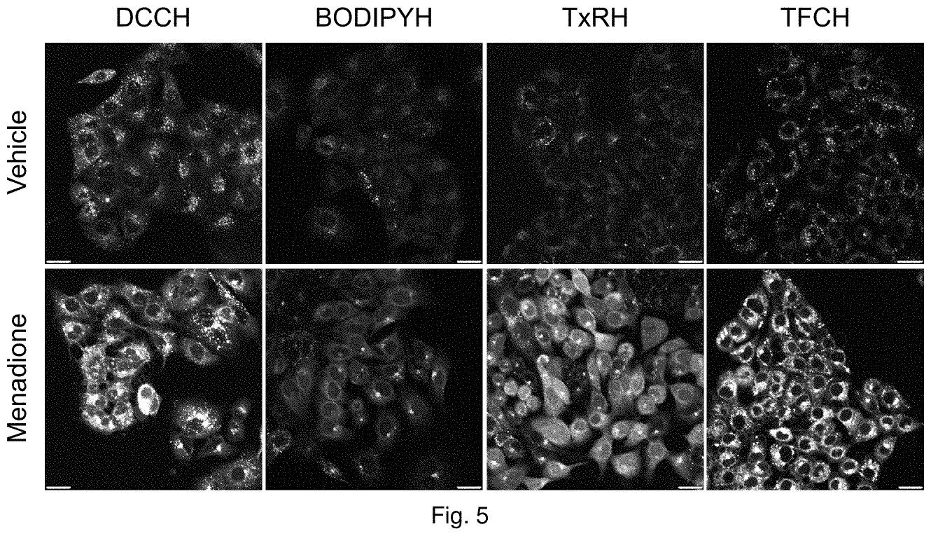

FIG. 5 shows results of menadione-induced carbonylation as detected in live cells. A549 cells were treated with vehicle (0.1% DMSO, v/v) or 100 μM menadione for 1 h, followed by 10 μM DCCH, BODIPYH, TxRH or TFCH for 30 min. The medium was discarded, and fresh medium was added to the cells and allowed to incubate for 5 min. After an additional round of medium change, the cells were imaged live using a confocal laser scanning microscope.

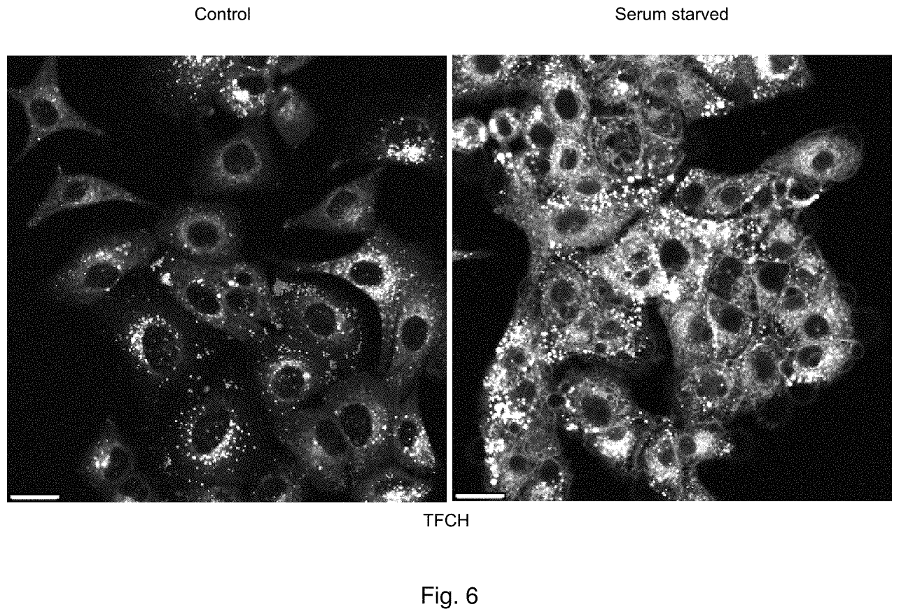

FIG. 6 shows results of serum starvation-induced carbonylation as detected in live cells by a one-step assay. A549 cells were grown in standard medium (control) or serum-free medium (serum starved) for 24 h. The medium was discarded and fresh medium containing 2 μM TFCH was added to the cells and incubated for 30 min before imaging using a confocal laser scanning microscope.

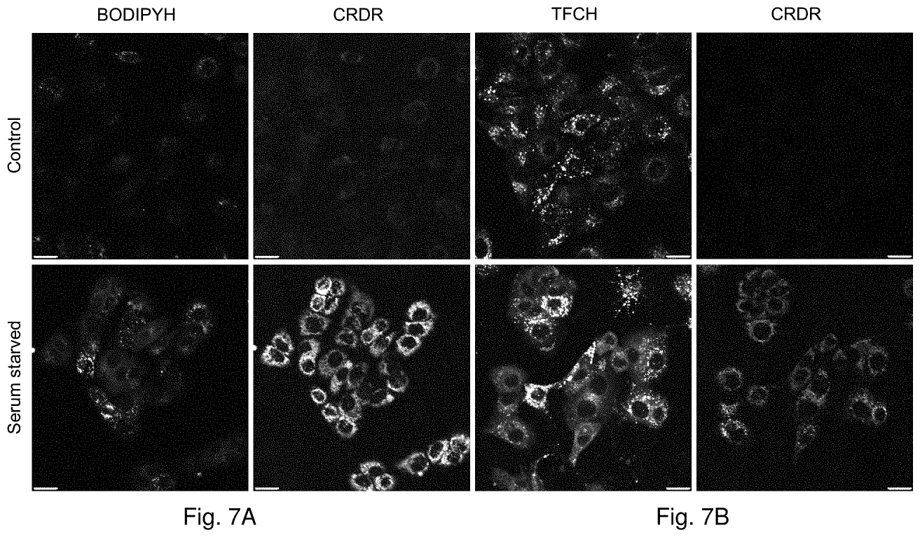

FIGS. 7A and 7B show results of a multiplex assay for carbonyls and ROS induced by serum starvation in live cells. A549 cells were grown in standard medium (control) or serum-free medium (serum starved) for 25 h. The medium was discarded and fresh medium containing 10 μM BODIPYH (A) or TFCH (B) and 5 μM CRDR was added to the cells and incubated for 30 min. The medium was discarded, and fresh medium was added to the cells and allowed to incubate for 5 min. After an additional round of medium change, the cells were imaged live using a confocal laser scanning microscope. Images from different emission channels were captured separately and then merged to produce the overlay images. Note: The emission window applied for CRDR detection in FIG. 7A (663-800 nm) was different from the emission window applied for CRDR in FIG. 7B (700-800 nm). The latter was reduced to prevent bleed-through from TFCH emission.

Oxidative stress-induced carbonylation and ROS in live cells (multiplexing) may be simultaneously detected. This protocol allows for observation of both OS-induced ROS and carbonylation simultaneously. CellROX™ Deep Red (CRDR) reagent was used in this protocol, which is compatible with BODIPYH (commercial) and TFCH (synthetic). At the end of the incubation period with the OSA, 1 μL of 2 mM fluorophore (final concentration of fluorophore=10 μM) and 0.4 μL of 2.5 mM ROS detection agent are added simultaneously (final concentration of CRDR=5 μM; final percent DMSO=0.7%, v/v) and mixed well by gently moving the chambered coverglass in a circular motion (clockwise as well as counter-clockwise), and then incubated for 30 min in the cell culture incubator. The medium is discarded and 500 μL fresh medium added to each well. The coverglass is returned to the cell culture incubator for 5 min. A single (longer) wash step may be employed to prevent premature release of adherent cells.

Another multiplex assay dye is resazurin, which is a phenoxazine dye that is weakly fluorescent, nontoxic, cell-permeable, and redox-sensitive. Resazurin has a blue to purple color (at pH>6.5) and is used in microbiological, cellular, and enzymatic assays because it can be irreversibly reduced to the pink-colored and highly fluorescent resorufin (7-Hydroxy-3H-phenoxazin-3-one). At circum-neutral pH, resorufin can be detected by visual observation of its pink color or by fluorimetry, with an excitation maximum at 530-570 nm and an emission maximum at 580-590 nm. When a solution containing resorufin is submitted to reducing conditions (Eh<−110 mV), almost all resorufin is reversibly reduced to the translucid non-fluorescent dihydroresorufin (sin. hydroresorufin) and the solution becomes translucid (the redox potential of the resorufin/dihydroresorufin pair is −51 mV vs. standard hydrogen electrode at pH 7.0). When the Eₕ of this same solution is increased, dihydroresorufin is oxidized back to resorufin, and this reversible reaction can be used to monitor if the redox potential of a culture medium remains at a sufficiently low level for anaerobic organisms. Resazurin is reduced to resorufin by aerobic respiration of metabolically active cells, and it can be used as an indicator of cell viability. It can be used to detect the presence of viable cells in mammalian cell cultures.

A further multiplex assay dye is sulforhodamine B (SRB), to measure drug-induced cytotoxicity and cell proliferation for large-scale drug-screening applications. Its principle is based on the ability of the protein dye sulforhodamine B to bind electrostatically and pH dependent on protein basic amino acid residues of trichloroacetic acid-fixed cells. Under mild acidic conditions it binds to and under mild basic conditions it can be extracted from cells and solubilized for measurement. See www.thermofisher.com/order/catalog/product/S1307?SID=srch-srp-S1307#/S1307?SID=srch-srp-S 1307

Similarly, the ROS-Glo™ H₂O₂ from Promega may be employed.

See, www.promega.com/resources/pubhub/a-luminescent-assay-for-detection-of-reactive-oxygen-species/

Molecular Probes also provides various reagents for assessing cells, e.g., CellROX® Deep Red, CellROX® Green and CellROX® Orange; CM-H₂-DCFDA; Image-It, MitoSOX Red Superoxide Indicator; Invitrogen Premo Cellular Hydrogen Peroxide H₂O₂ Sensor; Invitrogen Premo Cellular Redox Sensor, Grx-1-roGFP; ThiolTracker Violet dye; NucGreen Dead 488 ReadyProbes Reagent, NucRed Dead 647 ReadyProbes Reagent, ReadyProbes Cell Viability Imaging Kit (Blue/Green), ReadyProbes Cell Viability Imaging Kit (Blue/Red); See also, www.thermofisher.com/us/en/home/life-science/cell-analysis/cell-viability-and-regulation/oxidative-stress.html; www.thermofisher.com/search/browse/category/us/en/22501?navId=10364&persona=Catalog&resultsPerPage=60.

The multiplex assay may also employ 1,5-bis{[2-(di-methylamino) ethyl]amino}-4,8-dihydroxyanthracene-9,10-dione, DRAQ5 or DRAQ7. See, www.thermofisher.com/order/catalog/product/62251#/62251, or propidium iodide nuclear stain, see thermofisher.com/order/catalog/product/P1304MP?SID=srch-srp-P1304MP.

The cells fluorescently labeled with TFCH can be preserved for future imaging using a mountant. After preparing the samples as described above, the cells are rinsed with DPBS, and then treated with 4% paraformaldehyde in DPBS (PFA solution) for 15 min. The cells are then rinsed two more times with DPBS. The samples are mounted with ProLong™ Gold Antifade Mountant and cured overnight. The samples are stored at 4° C.

Imaging oxidative stress (OS) in live cells is traditionally performed using a fluorescent probe that undergoes reaction with reactive species that are produced during OS. An alternative to this method is presented in this chapter, which detects one of the biomarkers produced during OS, biomolecule carbonylation.

A preferred set of hydrazine-based fluorophores are designed to undergo a spectral change upon condensation with carbonyls.

The protocols here were tested using A549 cells, which have a low level of endogenous carbonylation (typical of cancer cells) and show a good response to serum starvation-induced OS. Since carbonylation in response to stressors varies with factors such as the cell line, the nature of the stressor, etc., alterations in incubation time and fluorophore concentration may be employed to optimize the results. Table 2 compares the washing method with the one-step method.

The present technology may be used to detect oxidative stress and its sequalae in cell culture, biopsy specimens, blood samples, and in intact tissues (typically sectioned for visualization). The technology may find particular application is assessing toxicity, such as in high throughput screening of drug candidates, clinical specimen assessment of ROS or other causes of carbonylation of biomolecules, and determination of bursts of cellular oxidative metabolism or other ROS, even where the condition has abated at the time of assessment.

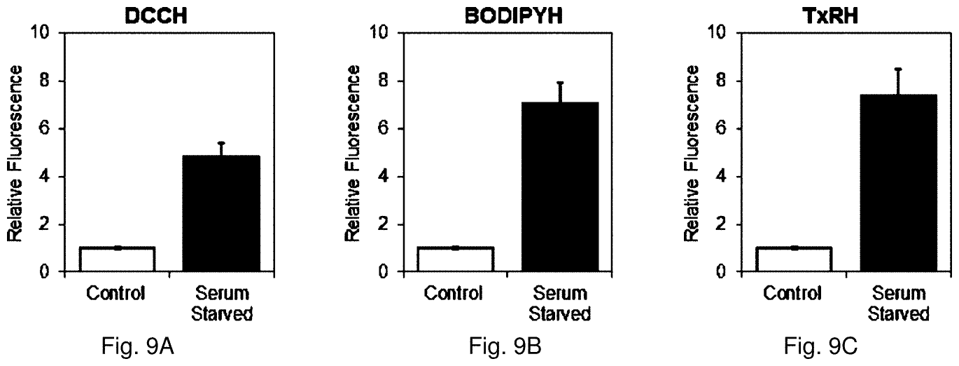

FIGS. 9A-9C show quantitative analysis of carbonyls in live cells in high throughput platform: A549 cells grown in standard media (control) or serum free media (serum starved) for ˜24 h were allowed to react with 10 μM hydrazide fluorophores for 1 h. Cells were washed and the emission of respective samples were recorded. The excitation (ex) and emission (em) wavelengths used are as follows: (FIG. 9A) DCCH (ex: 405 nm; em: 475 nm); (FIG. 9B) BODIPYH (ex: 488 nm; em: 530 nm); (FIG. 9C) TxRH (ex: 543 nm; em: 620 nm). Fluorescence in each sample was normalized to cell density. The graphs represent fluorescence of the serum starved samples relative to the control samples.

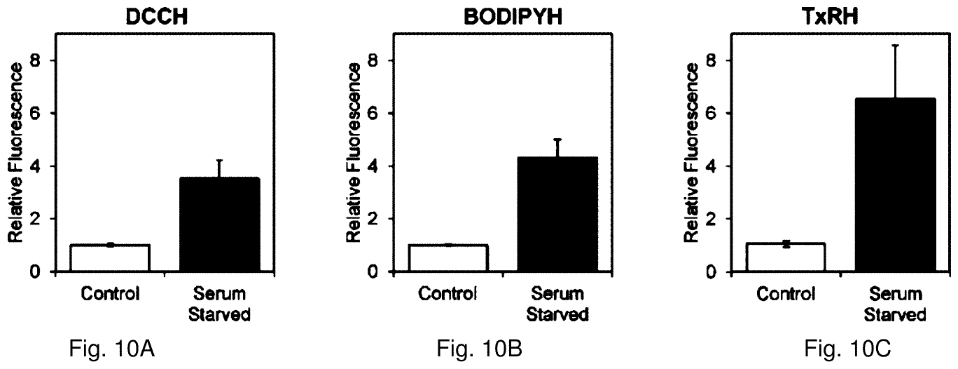

FIGS. 10A-10C show quantitative analysis of carbonyls in cells: A549 cells grown in standard media (control) or serum free media (serum starved) for 24-28 h were allowed to react with 10 82 M (FIG. 10A) DCCH, (FIG. 10B) BODIPYH or (FIG. 10C) TxRH for 1 h. Cells were washed, lysed and the emission of the respective samples were recorded. The excitation and emission wavelengths used are the same as FIGS. 9A-9C. Fluorescence in each sample was normalized to protein concentration representing cell density. The graphs represent fluorescence of the serum starved samples relative to the control samples.

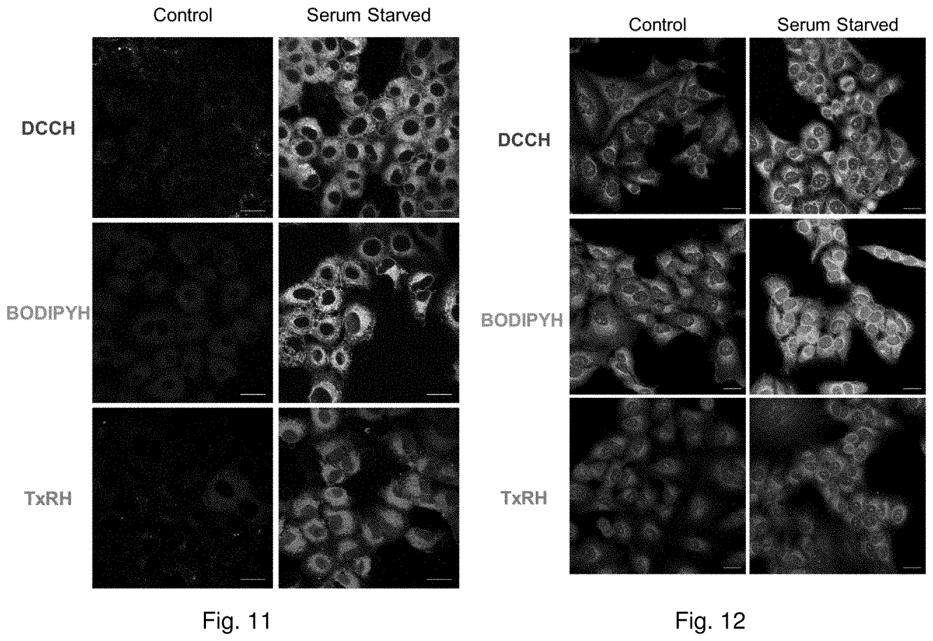

FIG. 11 shows a set of fluorescent confocal micrographs for detection and visualization of carbonyls in live cells: Control and serum starved A549 cells were treated with 20 μM hydrazide fluorophore for 30 min. The cells were washed and imaged live using a confocal microscope. The excitation lasers used are as follows: DCCH (ex: 405 nm); BODIPYH (ex: 488 nm) and TxRH (ex: 543 nm).

FIG. 12 shows a set of fluorescent confocal micrographs for detection and visualization of carbonyls in fixed A549 cells: Control and serum starved A549 cells were washed and fixed with methanol. After subsequent PBS rinse, the cells were incubated in serum free media with 10 μM hydrazide fluorophore for 3 h. The cells were washed with PBS and imaged using a confocal microscope. The excitation lasers used are as follows: DCCH (ex: 405 nm); BODIPYH (ex: 488 nm) and TxRH (ex: 543 nm). Table 1 shows criteria for selection of laser for confocal microscopy.

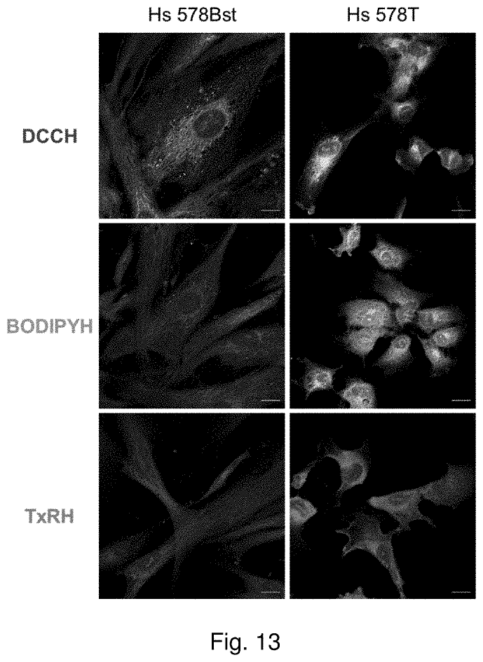

FIG. 13 shows a set of fluorescent confocal micrographs for detection and visualization of carbonyls in fixed Hs 578Bst (normal) and Hs 578T (carcinoma) cells: Hs 578Bst and Hs 578T cells were washed and fixed with methanol. The cells were processed and imaged as described in FIG. 12.

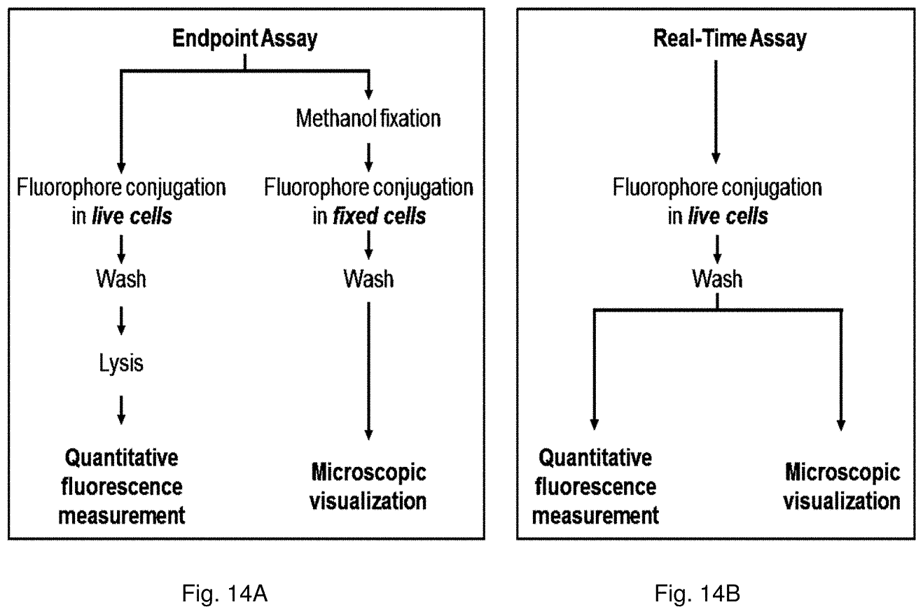

FIGS. 14A and 14B show a flow charts for cellular carbonyl detection assays. FIG. 14A shows an endpoint or fixed cell assays. FIG. 14B shows real-time or live cell assays.

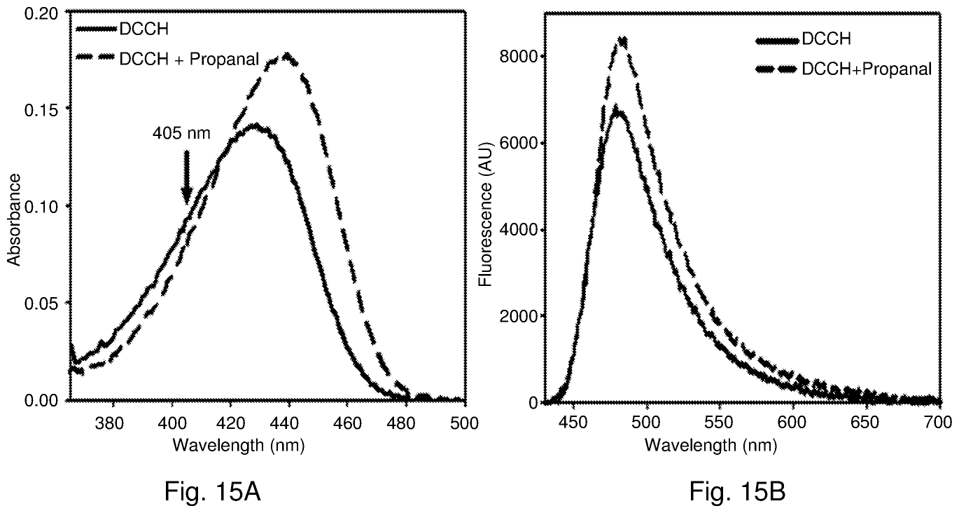

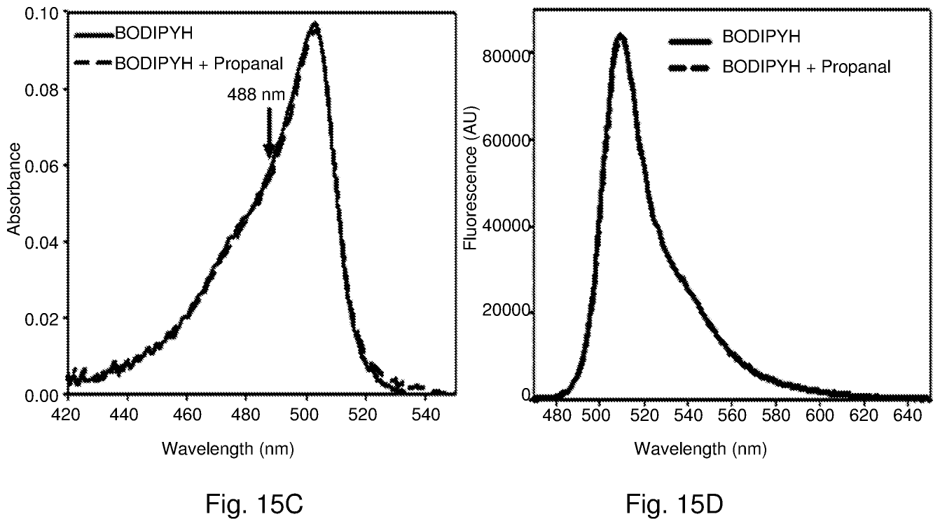

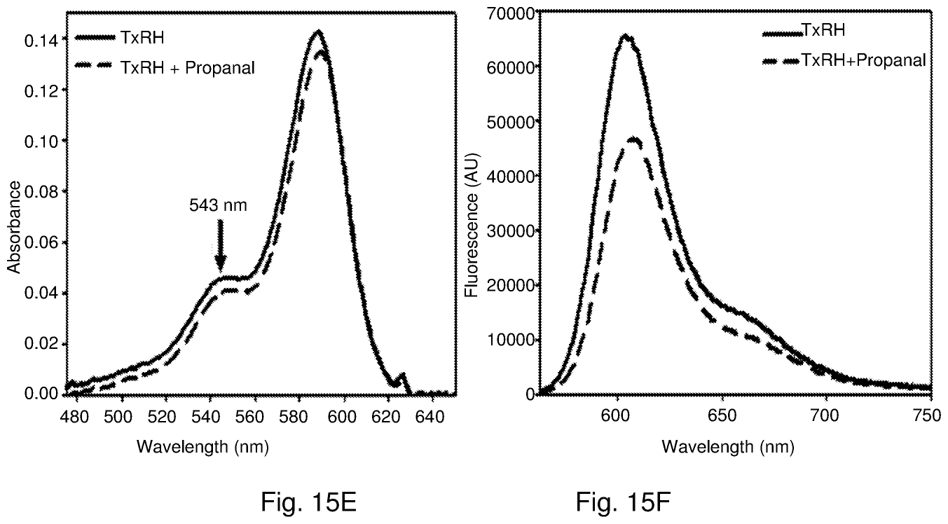

FIGS. 15A-15F show absorption (top) and emission (bottom) spectra of (FIGS. 15A, 15B) DCCH, (FIGS. 15C, 15D) BODIPYH, and (FIGS. 15E, 15F) TxRH and their corresponding hydrazones with propanal in 10 mM PB, pH 7 containing 0.5% DMSO (v/v). The excitation wavelengths used for obtaining the emission spectra are as follows: DCCH (ex: 405 nm), BODIPYH (ex: 450 nm), TxRH (543 nm). The arrow in the absorption spectra denotes the wavelength of the excitation laser used in the microscopy experiments. See Table 3.

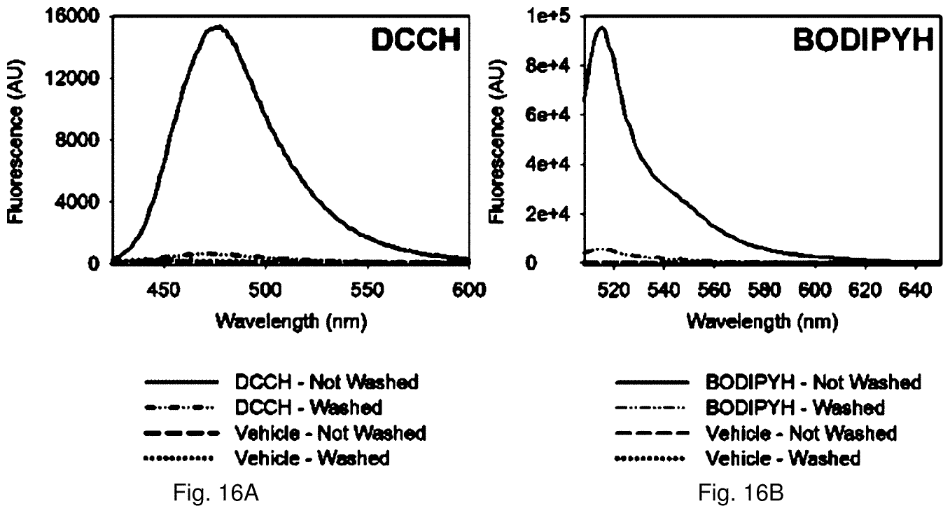

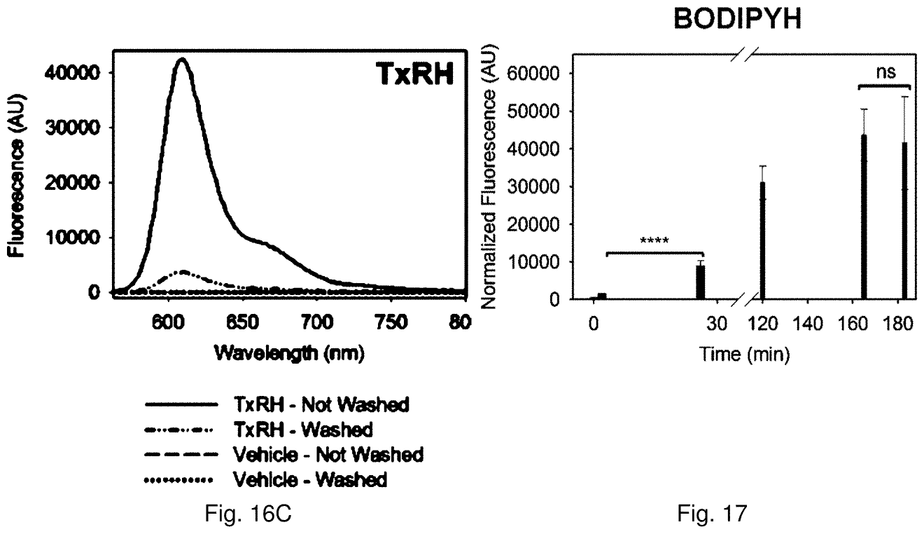

FIGS. 16A-16C show graphs of hydrazide fluorophore efflux from A549 cells: The cells were incubated with 20 μM fluorophore or vehicle (0.5% DMSO, v/v) for 5 min. The cells were either lysed immediately or washed (as described in methods) and lysed before recording the emission spectra. The excitation wavelengths used are as follows: (FIG. 16A) DCCH (ex: 405 nm), (FIG. 16B) BODIPYH (ex: 488 nm), (FIG. 16C) TxRH (543 nm).

FIG. 17 shows a time course of hydrazone formation in live A549 cells. Serum starved cells were allowed to react with 10 uM BODIPYH for the stated time before washing and acquiring emission at 520 nm (ex: 488 nm). Fluorescence in each sample was normalized to cell density. Two tailed unpaired t-test was performed to compare the emission between the indicated time points. ns=not significant, ****=p<0.0001.

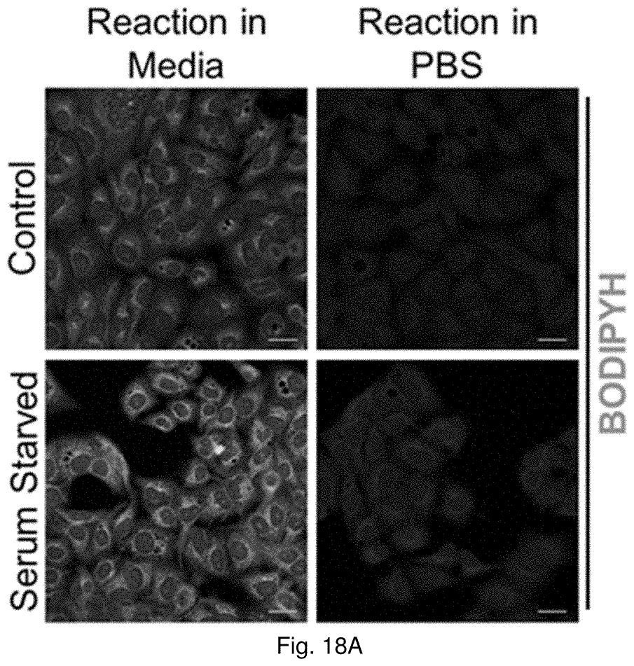

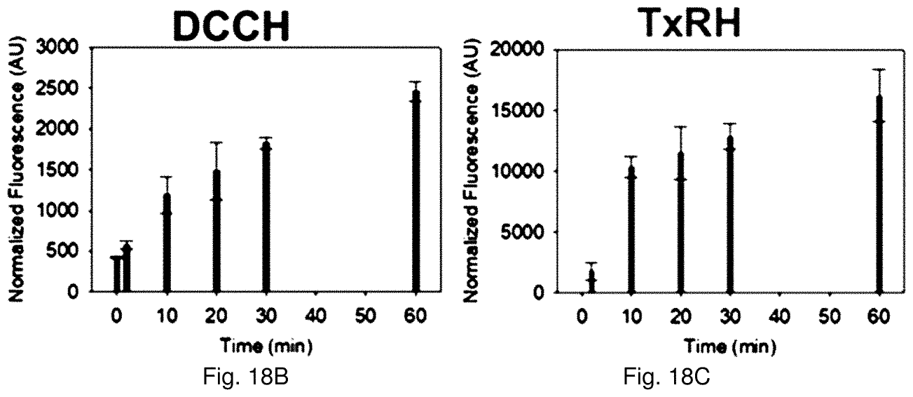

FIGS. 18A-18C show optimization of assay conditions for detecting and visualizing carbonyls in fixed cells. FIG. 18A shows a set of fluorescent confocal micrographs comparing PBS and serum free media to identify the optimal reaction media. Control and serum starved A549 cells were methanol-fixed (as described in methods) and allowed to react with 10 μM BODIPYH in PBS or serum free media for 1 h before washing and imaging. DCCH (FIG. 18B) or TxRH (10 μM) (FIG. 18C) were allowed to react with fixed serum starved cells for the stated time, washed and the emission was recorded using a platereader. Emission was normalized by cell density. The excitation (ex) and emission (em) wavelengths used are as follows: DCCH (ex: 405 nm; em: 475 nm); TxRH (ex: 543 nm; em: 620 nm).

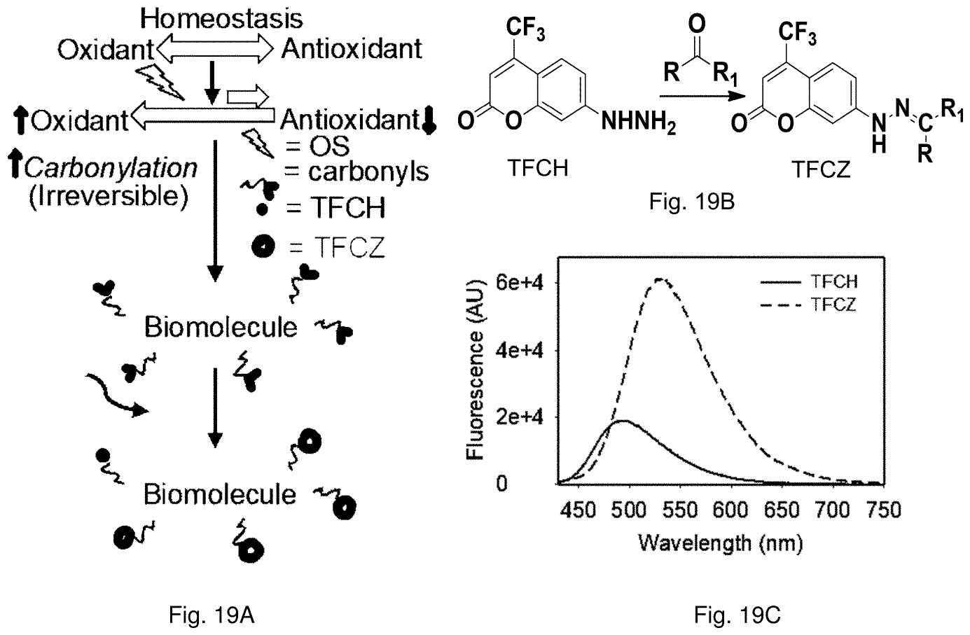

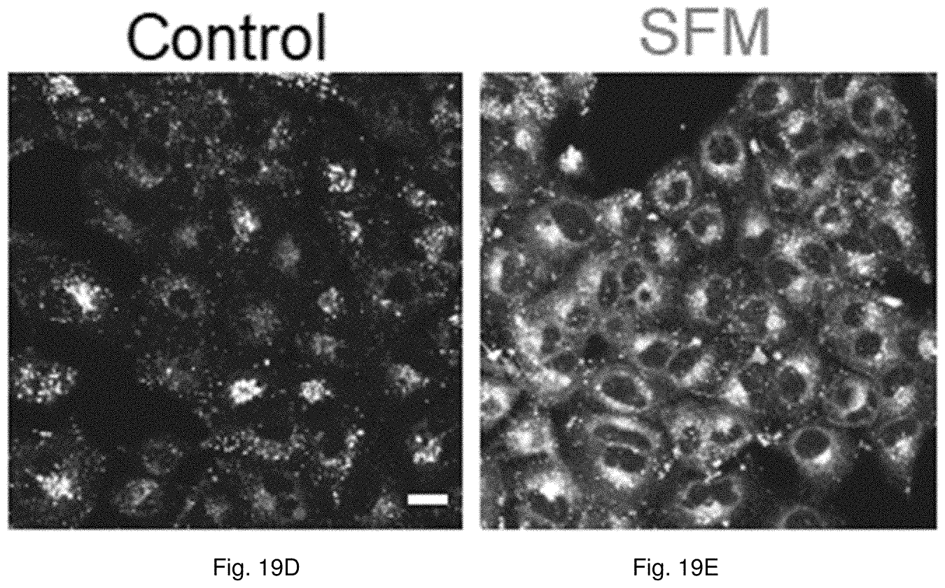

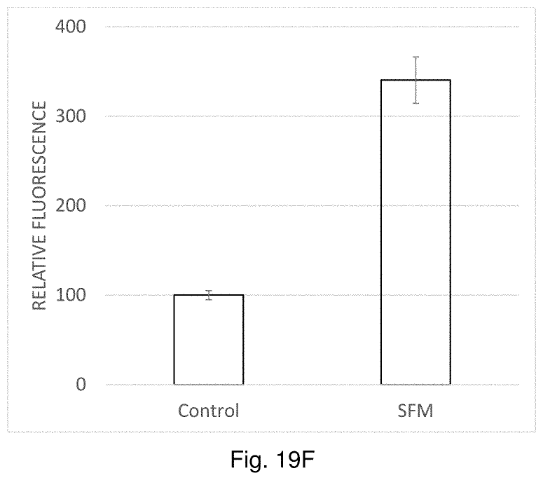

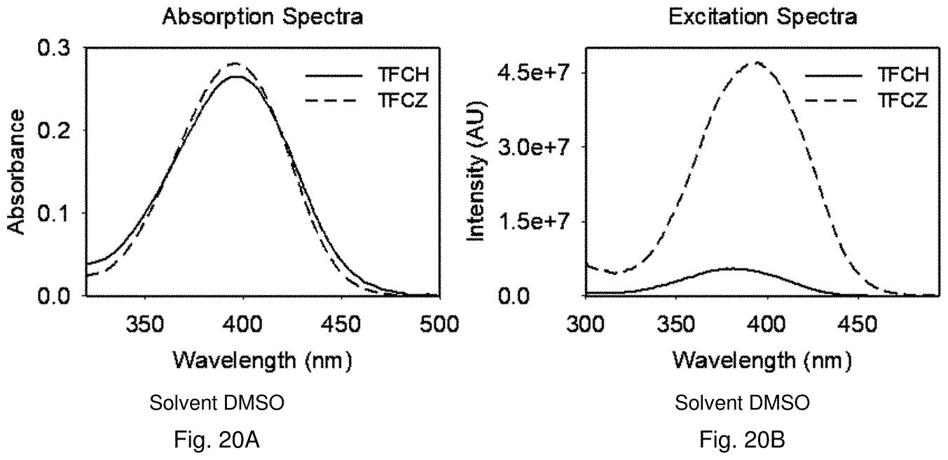

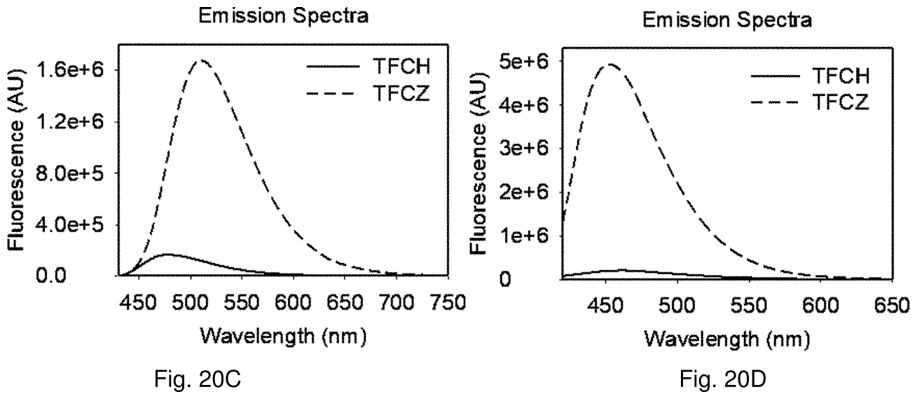

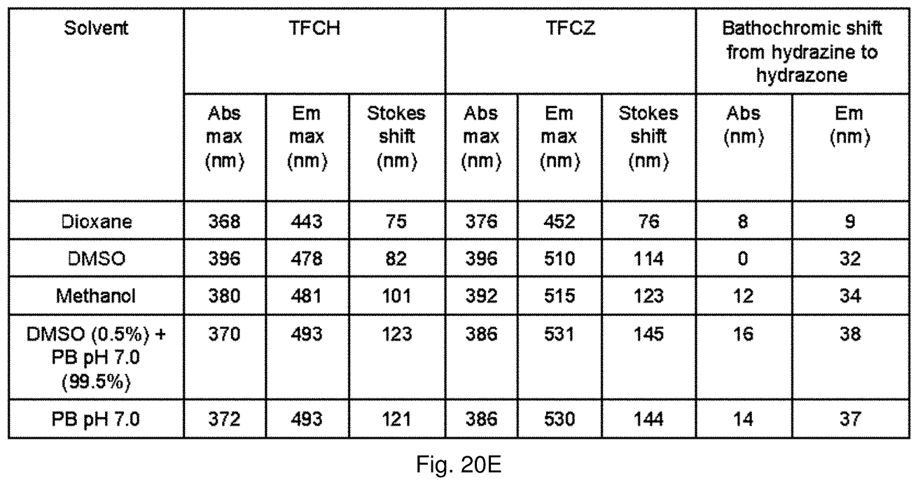

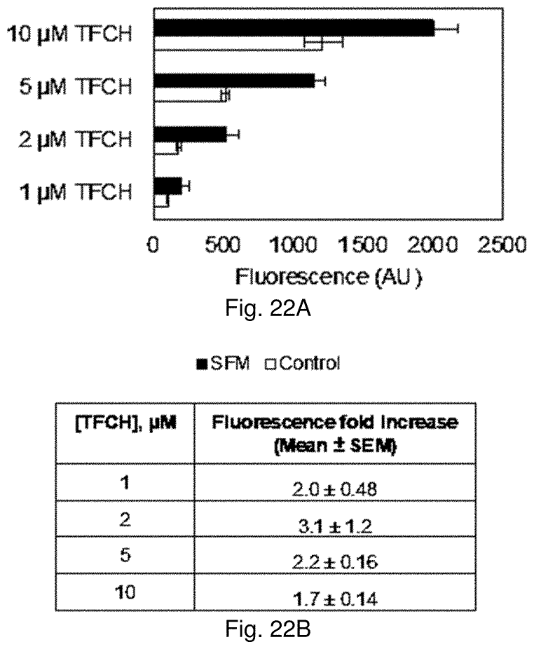

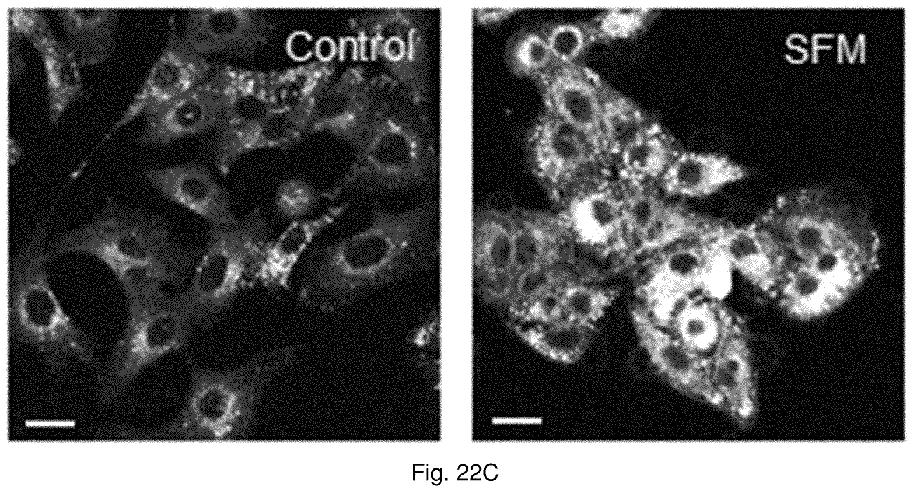

FIG. 19A shows a schematic representation of OS-induced carbonylation detected by TFCH. FIG. 19B shows hydrazone (TFCZ) formation. FIG. 19C shows emission spectra of TFCH and TFCZ (hydrazone of TFCH and propanal). FIGS. 19D-19F show that TFCH can detect SFM-induced carbonylation in live cells. FIGS. 20A-20E show photochemical properties of TFCH and TFCZ (hydrazone of TFCH and propanal). FIG. 22A shows absorption, FIG. 20B, excitation, and FIG. 20C, emission, spectra of 10 μM TFCH and TFCZ in DMSO. FIG. 20D shows emission spectra of TFCH and TFCZ in dioxane. FIG. 22E shows spectroscopic (absorption and emission) characterization of TFCH and TFCZ in different solvents.

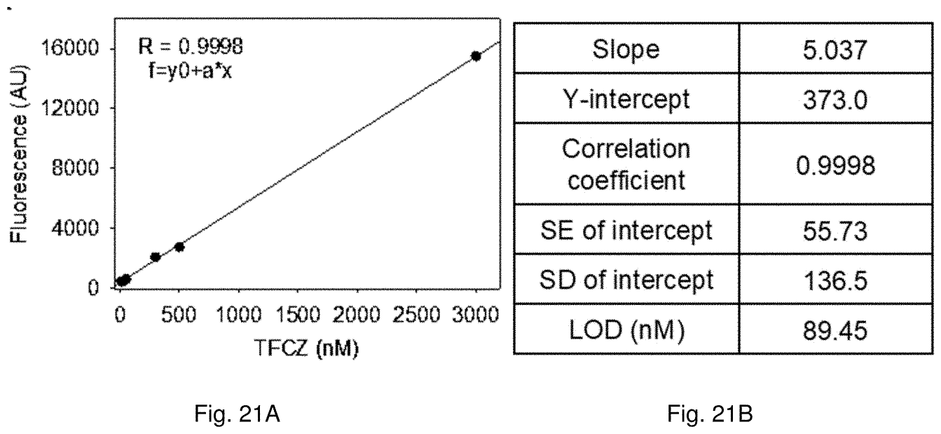

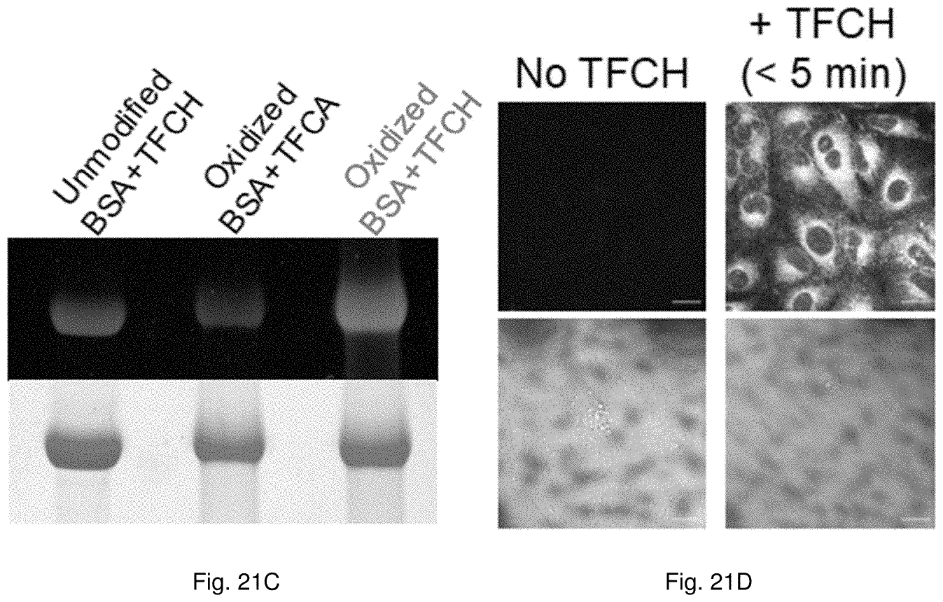

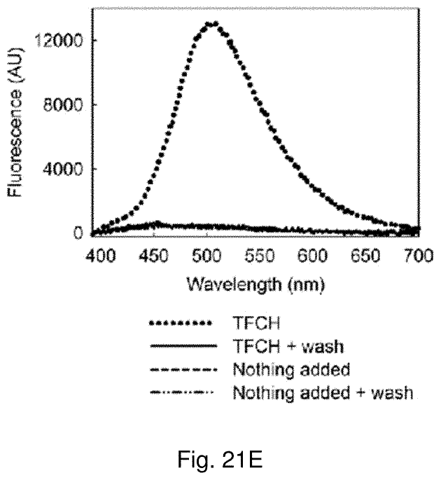

FIGS. 21A-21E show that TFCH is a live-cell compatible tool for detecting oxidative stress-induced carbonylation. FIGS. 21A-21B, Limit of detection (LOD) of TFCZ (hydrazone of TFCH and propanal) in A549 cell lysate. FIG. 21C shows that oxidized protein (oxidized BSA) can be detected by TFCH. Unmodified BSA reacted with TFCH and oxidized BSA reacted with TFCA (the amine derivative of TFC); these were included as controls. The upper panel shows the SDS-PAGE gel imaged under long-wavelength UV and the lower panel shows the Coomassie-stained gel. FIG. 21D shows that cellular influx of TFCH occurs seamlessly. Live A549 cells were imaged before (no TFCH) and <5 min after adding TFCH (+TFCH) to the cells. Scale bar, 20 μm. FIG. 21E shows that TFCH can be effectively washed out of live A549 cells. The cells were incubated with 20 μM TFCH for 5 min. They were then either lysed immediately, or washed and lysed. Emission spectra of the lysates were recorded by exciting the samples at 372 nm.

FIGS. 22A-24C show that TFCH serves as a tool for detecting oxidative stress-induced carbonylation in live cells by HCS- and HTS-compatible assays. FIGS. 22A-22B, a plate-reader based assay for detecting SFM-induced carbonylation. A549 cells were grown in standard media (control) or SFM for 24 h before adding the stated concentration of TFCH for 90 min. The cells were rinsed with PBS and the emission was recorded at 525 nm (excitation: 405 nm). FIG. 22A shows a graph showing relative fluorescence. FIG. 22B shows the fold-increase in fluorescence due to serum starvation. Error bars represent SEM. FIG. 22C shows a one-step assay for visualizing biomolecule-carbonyls in live cells. Serum-starved or control cells were allowed to react with 2 μM TFCH for 30 min before imaging the live cells without rinsing excess fluorophore. Scale bar, 20 μm.

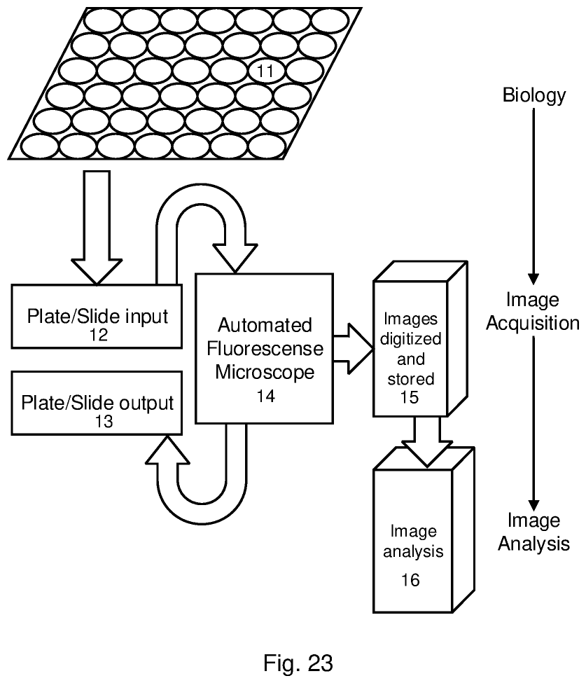

FIG. 23 shows a high throughput screening system. As shown in FIG. 23, a plate 11 of samples in an array is subject to biological experimentation, and then provided as a plate/slide input 12 to the automated fluorescence microscope 14. After image acquisition, images are digitized and stored 15, and the plate 11 is passed to the plate/slide output 13. Image analysis 16 is performed, yielding an automated data acquisition system which permits a large number of specimens to be analyzed with reduced manual effort.

One skilled in the art will readily appreciate that the specific methods and results discussed are merely illustrative of the invention as described more fully in the claims which follow thereafter.

TABLE 1
Recommended lasers and emission window for each condition

| | | Excitation | |
|---|---|---|---|
| Assay | Fluorophore | laser (nm) | Emission (nm) |
| Two-step assay | DCCH | 405 | LP 420 or 420-700 |
| (recommended) | BODIPYH | 488 | LP 505 or 505-650 |
| | TxRH | 543 | LP 560 or 560-700 |
| | TFCH | 405 | LP 420 or 420-700 |
| One-step assay | TFCH | 405 | LP 505 or 505-700 |
| (if significant | | | |
| cell detachment | | | |
| occurs due to | | | |
| oxidative stress) | | | |
| Multiplex assay | BODIPYH | 488 | 505-560 |
| | CRDR | 633 | 663-800 |
| | TFCH | 420 | 420-560 |
| | CRDR | 633 | 700-800 |

TABLE 2
Comparison of carbonylation detection tools

| | DCCH, BODIPYH, | |
|---|---|---|
| Attributes | TxRH | TFCH |
| Washing step to remove excess | ✓ | x |
| fluorophore required | | |
| One-step assay | x | ✓ |
| (no washing step required) | | |
| Compatible with commonly used laser | ✓ | ✓ |
| Compatible with commonly used | ✓ | ✓ |
| emission filters and monochromator | | |
| Flexibility in choosing colors | ✓ | x |
| Multiplexing | ✓ | ✓ |

*Other commercial hydrazides can be used; only BODIPYH was demonstrated experimentally.

TABLE 3
Absorption (abs) and emission (em) maxima of the hydrazide
fluorophores and their corresponding hydrazones with propanal.

| | Hydrazide | | Hydrazide + propanal | |
|---|---|---|---|---|
| | Abs max | Em max | Abs max | Em max |
| Fluorophores | (nm) | (nm) | (nm) | (nm) |
| DCCH | 429 | 479 | 439 | 481 |
| BODIPYH | 503 | 510 | 503 | 510 |
| TxRH | 589 | 607 | 589 | 607 |

### REFERENCES

Buss, H., Chan, T. P., Sluis, K. B., Domigan, N. M., & Winterbourn, C. C. (1997). Protein carbonyl measurement by a sensitive ELISA method. Free Radical Biology and Medicine, 23(3), 361-366. doi:10.1016/s0891-5849(97)00104-4

Chaudhuri, A. R., Wei, R., Bhattacharya, A., & Hamilton, R. (2015). Fluorescence-Based Approaches for Quantitative Assessment of Protein Carbonylation, Protein Disulfides, and Protein Conformation in Biological Tissues. In A. C. Shaw (Ed.), Immunosenescence. Methods in Molecular Biology (Vol. 1343, pp. 155-173). New York: Humana Press.

Chung, W. G., Miranda, C. L., & Maier, C. S. (2008). Detection of carbonyl-modified proteins in interfibrillar rat mitochondria using N′-aminooxymethylcarbonylhydrazino-D-biotin as an aldehyde/keto-reactive probe in combination with Western blot analysis and tandem mass spectrometry. Electrophoresis, 29(6), 1317-1324. doi:10.1002/elps.200700606

Coffey, C. M., & Gronert, S. (2016). A cleavable biotin tagging reagent that enables the enrichment and identification of carbonylation sites in proteins. Analytical and Bioanalytical Chemistry, 408(3), 865-874. doi:10.1007/s00216-015-9176-2

Colombo, G., Clerici, M., Garavaglia, M. E., Giustarini, D., Rossi, R., Milzani, A., & Dalle-Donne, I. (2016). A step-by-step protocol for assaying protein carbonylation in biological samples. Journal of Chromatography B: Analytical Technologies in the Biomedical and Life Sciences, 1019, 178-190. doi:10.1016/j.jchromb.2015.11.052

Dalle-Donne, I., Aldini, G., Carini, M., Colombo, R., Rossi, R., & Milzani, A. (2006).

Protein carbonylation, cellular dysfunction, and disease progression. Journal of Cellular and Molecular Medicine, 10(2), 389-406. doi:10.1111/j.1582-4934.2006.tb00407.x

Dalle-Donne, I., Giustarini, D., Colombo, R., Rossi, R., & Milzani, A. (2003). Protein carbonylation in human diseases. Trends in Molecular Medicine, 9(4), 169-176. doi:S1471491403000315 [pii]

Dalle-Donne, I., Rossi, R., Colombo, R., Giustarini, D., & Milzani, A. (2006). Biomarkers of oxidative damage in human disease. Clinical Chemistry, 52(4), 601-623. doi:10.1373/clinchem.2005.061408

Frohnert, B. I., & Bernlohr, D. A. (2013). Protein carbonylation, mitochondrial dysfunction, and insulin resistance. Advances in Nutrition, 4(2), 157-163. doi:10.3945/an.112.003319

Georgiou, C. D., Zisimopoulos, D., Argyropoulou, V., Kalaitzopoulou, E., Ioannou, P. V., Salachas, G., & Grune, T. (2018). Protein carbonyl determination by a rhodamine B hydrazide-based fluorometric assay. Redox Biology, 17, 236-245. doi:10.1016/j.redox.2018.04.017

Halliwell, B., & Chirico, S. (1993). Lipid peroxidation: its mechanism, measurement, and significance. American Journal of Clinical Nutrition, 57(5 Suppl), 715S-724S; discussion 724S-725S. doi:10.1093/ajcn/57.5.715S

Hauck, A. K., & Bernlohr, D. A. (2016). Oxidative stress and lipotoxicity. Journal of Lipid Research, 57(11), 1976-1986. doi:10.1194/j1r.R066597

Katerji, M., Filippova, M., & Duerksen-Hughes, P. (2019). Approaches and Methods to Measure Oxidative Stress in Clinical Samples: Research Applications in the Cancer Field. Oxidative medicine and cellular longevity, 2019, 29. doi:10.1155/2019/1279250

Kölmel, D. K., & Kool, E. T. (2017). Oximes and Hydrazones in Bioconjugation: Mechanism and Catalysis. Chemical Reviews, 117(15), 10358-10376. doi:10.1021/acs.chemrev.7b00090

Kuzmic, M., Javot, H., Bonzom, J. M., Lecomte-Pradines, C., Radman, M., Gamier-Laplace, J., & Frelon, S. (2016). In situ visualization of carbonylation and its co-localization with proteins, lipids, DNA and RNA in Caenorhabditis elegans. Free Radical Biology and Medicine, 101, 465-474. doi:10.1016/j.freeradbiomed.2016.11.004

Levine, R. L., Garland, D., Oliver, C. N., Amici, A., Climent, I., Lenz, A. G., . . . Stadtman, E. R. (1990). Determination of Carbonyl Content in Oxidatively Modified Proteins. Methods in Enzymology, 186, 464-478.

Lucero, H. A., Ravid, K., Grimsby, J. L., Rich, C. B., DiCamillo, S. J., Maki, J. M., . . . Kagan, H. M. (2008). Lysyl oxidase oxidizes cell membrane proteins and enhances the chemotactic response of vascular smooth muscle cells. Journal of Biological Chemistry, 283(35), 24103-24117. doi:10.1074/jbc.M709897200

Madian, A. G., & Regnier, F. E. (2010). Profiling carbonylated proteins in human plasma. J Proteome Res, 9(3), 1330-1343. doi:10.1021/pr900890k

Marnett, L. J. (2000). Oxyradicals and DNA damage. Carcinogenesis, 21(3), 361-370. doi:10.1093/carcin/21.3.361

Mesquita, C. S., Oliveira, R., Bento, F., Geraldo, D., Rodrigues, J. V., & Marcos, J. C. (2014). Simplified 2,4-dinitrophenylhydrazine spectrophotometric assay for quantification of carbonyls in oxidized proteins. Analytical Biochemistry, 458, 69-71. doi:10.1016/j.ab.2014.04.034

Mukherjee, K., Chio, T. I., Gu, H., Banerjee, A., Sorrentino, A. M., Sackett, D. L., & Bane, S. L. (2017). Benzocoumarin Hydrazine: A Large Stokes Shift Fluorogenic Sensor for Detecting Carbonyls in Isolated Biomolecules and in Live Cells. ACS Sensors, 2(1), 128-134. doi:10.1021/acssensors.6b00616

Mukherjee, K., Chio, T. I., Sackett, D. L., & Bane, S. L. (2015). Detection of oxidative stress-induced carbonylation in live mammalian cells. Free Radical Biology and Medicine, 84, 11-21. doi:10.1016/j.freeradbiomed.2015.03.011

Nascimento, C., Kim, H. K., Young, L. T., Mendonca, K. M., Grinberg, L. T., Lafer, B., & Andreazza, A. C. (2015). Glutathione-mediated effects of lithium in decreasing protein oxidation induced by mitochondrial complex I dysfunction. Journal of Neural Transmission, 122(6), 741-746. doi:10.1007/s00702-014-1318-8

Ramana, K. V., Srivastava, S., & Singhal, S. S. (2014). Lipid peroxidation products in human health and disease 2014. Oxidative Medicine and Cellular Longevity, 2014, 162414. doi:10.1155/2014/162414

Talarico, C., Dattilo, V., D'Antona, L., Barone, A., Amodio, N., Belviso, S., . . . Amato, R. (2016). SI113, a SGK1 inhibitor, potentiates the effects of radiotherapy, modulates the response to oxidative stress and induces cytotoxic autophagy in human glioblastoma multiforme cells. Oncotarget, 7(13), 15868-15884. doi:10.18632/oncotarget.7520

Tamarit, J., de Hoogh, A., Obis, E., Alsina, D., Cabiscol, E., & Ros, J. (2012). Analysis of oxidative stress-induced protein carbonylation using fluorescent hydrazides. Journal of Proteomics, 75(12), 3778-3788. doi:10.1016/j.jprot.2012.04.046

Thanan, R., Oikawa, S., Hiraku, Y., Ohnishi, S., Ma, N., Pinlaor, S., . . . Murata, M. (2014). Oxidative stress and its significant roles in neurodegenerative diseases and cancer. International Journal of Molecular Sciences, 16(1), 193-217. doi:10.3390/ijms16010193

Ugur, Z., Coffey, C. M., & Gronert, S. (2012). Comparing the efficiencies of hydrazide labels in the study of protein carbonylation in human serum albumin. Analytical and Bioanalytical Chemistry, 404(5), 1399-1411. doi:10.1007/s00216-012-6235-9

Vemula, V., Ni, Z., & Fedorova, M. (2015). Fluorescence labeling of carbonylated lipids and proteins in cells using coumarin-hydrazide. Redox Biology, 5, 195-204. doi:10.1016/j.redox.2015.04.006

Wang, X., Fang, H., Huang, Z., Shang, W., Hou, T., Cheng, A., & Cheng, H. (2013). Imaging ROS signaling in cells and animals. Journal of molecular medicine (Berlin, Germany), 91(8), 917-927. doi:10.1007/s00109-013-1067-4

Wang, Xin, Ping Li, Qi Ding, Chuanchen Wu, Wen Zhang, and Bo Tang. “Illuminating the Function of the Hydroxyl Radical in the Brains of Mice with Depression Phenotypes by Two-Photon Fluorescence Imaging.” Angewandte Chemie 131, no. 14 (2019): 4722-4726.

Weber, D., Davies, M. J., & Grune, T. (2015). Determination of protein carbonyls in plasma, cell extracts, tissue homogenates, isolated proteins: Focus on sample preparation and derivatization conditions. Redox Biology, 5, 367-380. doi:10.1016/j.redox.2015.06.005

Wehr, N. B., & Levine, R. L. (2013). Quantification of protein carbonylation. Methods in Molecular Biology, 965, 265-281. doi:10.1007/978-1-62703-239-1_18

Yan, L.-J., & Forster, M. J. (2011). Chemical probes for analysis of carbonylated proteins: a review. Journal of chromatography. B, Analytical technologies in the biomedical and life sciences, 879(17-18), 1308-1315. doi:10.1016/j.jchromb.2010.08.004

Yan, L. J., & Forster, M. J. (2011). Chemical probes for analysis of carbonylated proteins: a review. Journal of Chromatography B: Analytical Technologies in the Biomedical and Life Sciences, 879(17-18), 1308-1315. doi:10.1016/j jchromb.2010.08.004

Yoo, B. S., & Regnier, F. E. (2004). Proteomic analysis of carbonylated proteins in two-dimensional gel electrophoresis using avidin-fluorescein affinity staining. Electrophoresis, 25(9), 1334-1341. doi:10.1002/elps.200405890

Zhang, Y., Dai, M., & Yuan, Z. (2018). Methods for the detection of reactive oxygen species. Analytical Methods, 10(38), 4625-4638. doi:10.1039/C8AY01339J

Dilek, O., Mukherjee, K. and Bane, S., 2012, January. Fluorescent Probes for Carbonyl-Containing Proteins. In Molecular Biology Of The Cell (Vol. 23). 8120 Woodmont Ave, Ste 750, Bethesda, Md. 20814-2755 USA: Amer Soc Cell Biology.

Mukherjee, K., Chio, T. I., Sorrentino, A. M., Sackett, D. L. and Bane, S. L., 2014, December. One Step Detection of Oxidative Stress-Induced Carbonylation in Live Mammalian Cells. In Molecular Biology Of The Cell (Vol. 25). 8120 Woodmont Ave, Ste 750, Bethesda, Md. 20814-2755 USA: Amer Soc Cell Biology.

Mukherjee, K., Chio, T. I., Sackett, D. L., and Bane, S. L., 2015. Comparative Analysis of Hydrazine-and Hydrazide-Functionalized Optical Probes for the Detection of Oxidative Stress Induced Biomolecule Carbonylation in Live Cells. Free Radical Biology and Medicine, 1(87), p.S151. www.sciencedirect.com/science/article/abs/pii/S089158491501000X?via%3Dihub

Ghosh, S., Mukherjee, K., Sackett, D. L. and Bane, S. L., 2017, January. Development of a new photo-stable fluorescence probe for detecting biomolecule carbonylation in live mammalian cells. In Molecular Biology Of The Cell (Vol. 28). 8120 Woodmont Ave, Ste 750, Bethesda, Md. 20814-2755 USA: Amer Soc Cell Biology.

Mukherjee, K., Chio, T., Sorrentino, A., Sackett, D. and Bane, S., 2017, December. Fluorogenic Sensors for Carbonylation in Live Cells. In Protein Science (Vol. 26, Pp. 44-44). 111 River St, Hoboken 07030-5774, N.J. USA: Wiley. www.ncbi.nlm.nih.gov/pmc/articles/PMC5743843/

Mukherjee, K., Chio, T., Sackett, D. and Bane, S., 2018, November. Fluorescent Sensors for Detection of Protein Carbonylation. In Protein Science (Vol. 27, pp. 73-74). 111 River St, Hoboken 07030-5774, N.J. USA: Wiley. onlinelibrary.wiley.com/doi/full/10.1002/pro.3513

Mukherjee, K., Chio, T. I., Sackett, D. L., and Bane, S. L., 2015. Detection of oxidative stress-induced carbonylation in live mammalian cells. Free Radical Biology and Medicine, 84, pp.11-21. www.sciencedirect.com/science/article/abs/pii/50891584915001239

Mukherjee, K., Chio, T. I., Gu, H., Banerjee, A., Sorrentino, A. M., Sackett, D. L. and Bane, S. L., 2017. Benzocoumarin Hydrazine: A Large Stokes Shift Fluorogenic Sensor for Detecting Carbonyls in Isolated Biomolecules and in Live Cells. ACS sensors, 2(1), pp.128-134. pubs.acs.org/doi/abs/10.1021/acssensors.6b00616

What is claimed is:

1. A method of detecting carbonylated biomolecules in living cells, comprising:
incubating the living cells with a fluorophore having at least one of a hydrazine group, an alkoxyamine group, and a hydrazide group;
reacting the fluorophore with carbonylated biomolecules of the living cells to couple the fluorophore to the biomolecules through a hydrazone or oxime linkage; and
detecting the fluorophore by imaging under illumination of the fluorophore reacted with the carbonylated biomolecules, having a limit of detection (LOD) in cell lysate of less than 100 nM.

2. The method according to claim 1, wherein the fluorophore having at least one of a hydrazine group or a hydrazide group comprises 7-hydrazinyl-4-trifluoromethylcoumarin (TFCH).

4. The method according to claim 1, wherein the fluorophore reacted with the carbonylated biomolecules comprises 7-hydrazinyl-4-trifluoromethylcoumarin hydrazone (TFCZ).

5. The method according to claim 1, wherein the fluorophore has a fluorescent emission peak which shifts as a result of the hydrazone or oxime formation,
further comprising analyzing the detected fluorophore to at least one of:
distinguish between cancer cells and normal cells;
assess cell aging and senescence; and
assessing a toxic insult to the cells,
based on a level of carbonylation of biomolecules in the living cells.

6. The method according to claim 1, wherein the linkage is a hydrazone.

7. The method according to claim 1, wherein the linkage is an oxime.

6. The method according to claim 1, wherein imaging is performed on unwashed living cells using at least one of 2-photon microscopy and confocal microscopy.

7. The method according to claim 1, wherein the fluorophore coupled to the biomolecules through the hydrazone or oxime linkage comprises a 7-hydrazinyl, 7-alkoxyamine or 7-hydrazide coumarin derivative having a 4-nucleophilic substituent, having a quantum yield of greater than 0.1 in 0.5% DMSO in phosphate buffered water at pH 7.0.

8. A method of detecting carbonylated macromolecules in body fluids or cells, comprising:
incubating the body fluid or cells having the carbonylated macromolecules with a fluorophore comprising a 7-hydrazinyl, 7-alkoxyamine or 7-hydrazide coumarin derivative having a 4-nucleophilic substituent to form a fluorophore conjugated to the carbonylated macromolecules through a hydrazone or oxime linkage;
illuminating the conjugated fluorophore at a first wavelength; and
detecting the conjugated fluorophore by imaging fluorescent emissions at a second wavelength within 90 minutes of commencement of the incubating, wherein fluorescent emissions of the fluorophore at the second wavelength are substantially non-interfering.

9. The method according to claim 8, wherein the conjugated fluorophore has a quantum yield of greater than 0.1 in 0.5% DMSO in phosphate buffered water at pH 7.0.

10. The method according to claim 8, wherein the cells are living cells, further comprising exposing the living cells to a drug prior to incubating, wherein the carbonylated macromolecules result from reactive oxygen species caused by metabolism of the drug by the living cells, and the detecting comprises quantifying free radical-induced oxidative damage to the living cells from the drug.

11. The method according to claim 8, wherein the fluorophore comprises 7-hydrazinyl 4-trifluoromethyl coumarin.

12. The method according to claim 8, wherein the detecting is concurrent with fluorescent imaging of a distinct fluorophore at a different wavelength from the second wavelength.

13. The method according to claim 12, wherein the distinct fluorophore is selected from the group consisting of resazurin, sulforhodamine B, and pyridium iodide, propidium iodide, and a 1,5-bis{[2-(di-methylamino) ethyl]amino}-4,8-dihydroxyanthracene-9,10-dione.

14. The method according to claim 8, wherein the incubating comprises providing a multiwell plate having a plurality of wells, each well having living cells in a medium, and adding a different drug treatment to different subsets of the plurality of wells.

15. A reaction product of a fluorophore comprising a 7-hydrazinyl, 7-alkoxyamine, or 7-hydrazide, 4-nucleophilic substituted coumarin, and at least one of a carbonylated lipid, a carbonylated nucleic acid, a carbonylated carbohydrate, and a carbonylated polypeptide.

16. The reaction product according to claim 15, wherein the 7-hydrazinyl 4-nucleophilic substituted coumarin derivative is 7-hydrazinyl-4-trifluoromethylcoumarin (TFCH).

17. The reaction product according to claim 15, wherein the 7-alkoxyamine 4-trifluoromethylcoumarin (TFCH).

18. The reaction product according to claim 15, wherein the 7-hydrazide 4-trifluoromethylcoumarin (TFCH).

19. The reaction product according to claim 15, wherein the fluorophore has a 3-substituent other than hydrogen.

20. The reaction product according to claim 15, wherein the fluorophore has a quantum yield of greater than 0.1 in 0.5% DMSO in phosphate buffered water at pH 7.0.
